(19) United States
(12) Patent Application Publication
(10) Pub. No.: US 2022/0288241 A1
(43) Pub. Date: Sep. 15, 2022

(54) LARGE SCALE PRODUCTION OF EXOSOME MIMETICS AND USES THEREOF

(71) Applicant: Children's Medical Center Corporation, Boston, MA (US)

(72) Inventors: Marsha A. Moses, Brookline, MA (US); Peng Guo, Boston, MA (US); Golnaz Morad, Boston, MA (US); Jing Huang, Natick, MA (US)

(73) Assignee: Children's Medical Center Corporation, Boston, MA (US)

(21) Appl. No.: 17/635,409

(22) PCT Filed: Aug. 13, 2020
(86) PCT No.: PCT/US2020/046050

§ 371 Date: Feb. 15, 2022

Related U.S. Application Data
(60) Provisional application No. 62/887,876, filed on Aug. 16, 2019.

(51) Int. Cl. A61K 49/18; A61K 9/50

(52) U.S. Cl. CPC A61K 49/1896; A61K 9/5068

(57) ABSTRACT

Provided herein are methods of producing exosome mimetics that are homogenous in size and are akin to native exosomes in structure and/or biological function. Also provided herein are the use of the EMs as delivery vehicles to deliver agents (e.g., therapeutic agents or diagnostic agents) for treating or diagnosing a disease.

### RELATED APPLICATION

This Application claims the benefit under 35 U.S.C. § 119(e) of U.S. Provisional Application Serial No. 62/887,876, entitled “LARGE SCALE PRODUCTION OF EXOSOME MIMETICS AND USES THEREOF” filed on Aug. 16, 2019, the entire contents of which is incorporated herein by reference.

### GOVERNMENT SUPPORT

This invention was made with government support under grant number CA185530 awarded by the National Institutes of Health. The Government has certain rights in the invention.

### BACKGROUND

Cell-derived extracellular vesicles (EVs) such as exosomes have been used as native drug delivery tools in the diagnosis and treatment of a variety of diseases. EVs are advantageous as delivery vehicles due to a variety of benefits such as lack of immunogenicity and ability to efficiently home to different organs. However, a robust and reproducible method for large scale production of exosomes is lacking. Conventional methods for isolating exosomes (e.g., ultracentrifugation) are low-efficient, time-consuming, and frequently require expensive instruments.

### SUMMARY

Described herein, in some aspects, are novel magnetic extrusion methods of producing endosome-derived nanoscale vesicles (termed herein “exosome mimetics (EMs)”) from different cultured cell lines in a large-scale and reproducible manner. The EMs produced using the methods described herein exhibit similar biological functions as the native exosomes. In some embodiments, therapeutic agents are encapsulated into engineered EMs with high encapsulation efficiency (e.g., more than 95%, more that 90%, more than 85%, more than 80%, more than 75%, or more than 70%). The methods described herein can be used for industrial production of GMP-grade exosome-based drug delivery systems. In some embodiments, the EMs produced using the methods described herein can be used for delivery of agents (e.g., therapeutic agents or diagnostic agents) for the treatment or diagnosis of a wide variety of diseases.

Accordingly, some aspects of the present disclosure provide methods of producing an exosome mimetic, the method comprising: (i) incubating a cell with a magnetic nanoparticle such that the magnetic nanoparticle enters an endosome in the cell; (ii) lysing the cell to produce a cell lysate containing the endosome; (iii) isolating the endosome encapsulating the magnetic nanoparticle from the cell lysate in step (ii); and (iv) extruding the isolated endosome obtained in step (iii) through a nanoporous membrane to produce the exosome mimetic.

In some embodiments, the cell is selected from stem cells, bone marrow derived cells, immune cells, red blood cells, epithelial cells, stem cells, and endothelial cells.

In some embodiments, the magnetic nanoparticle is an iron oxide nanoparticle.

In some embodiments, the nanoparticle enters the endosome in the cell via endocytosis.

In some embodiments, the cell is lysed via homogenization.

In some embodiments, step (iii) is carried out using a magnetic separator.

In some embodiments, the nanoporous membrane has a pore diameter of 100 nm.

In some embodiments, the method further comprises: (v) removing unencapsulated magnetic nanoparticles. In some embodiments, step (v) is carried out via size exclusion chromatography.

In some embodiments, the method furthering comprises: (vi) removing the magnetic nanoparticle from the exosome mimetic.

In some embodiments, the magnetic nanoparticle is conjugated to a targeting moiety, a therapeutic agent, or a diagnostic agent.

Other aspects of the present disclosure provide exosome mimetics produced by the methods described herein. In some embodiments, the exosome mimetic comprises a magnetic nanoparticle. In some embodiments, the exosome mimetic further comprises an agent. In some embodiments, the agent is a therapeutic agent or a diagnostic agent. In some embodiments, the agent is conjugated to the magnetic nanoparticle. In some embodiments, the magnetic nanoparticle is an iron oxide nanoparticles.

Further provided are compositions comprising the exosome mimetic described herein. In some embodiments, the composition further comprises a pharmaceutically acceptable carrier.

Other aspects of the present disclosure relate to methods of treating a disease, the method comprising administering to a subject in need thereof an effective amount of the exosome mimetic or the composition described herein. In some embodiments, the disease is: cancer, cardiovascular diseases, brain diseases, immune deficiency, autoimmune and infectious diseases, respiratory diseases, or endocrine system diseases.

Other aspects of the present disclosure relate to methods of diagnosing a disease, the method comprising administering to a subject in need thereof an effective amount of the exosome mimetic or the composition described herein, wherein the exosome mimetic comprises a diagnostic agent. In some embodiments, the disease is: cancer, cardiovascular diseases, brain diseases, immune deficiency, autoimmune and infectious diseases, respiratory diseases, or endocrine system diseases.

Further provided herein are in vivo imaging methods, the methods comprising administering to a subject in need thereof an effective amount of the exosome mimetic described herein and visualizing the exosome mimetic in the subject via magnetic resonance imaging (MRI), fluorescent imaging, PET imaging, bioluminescence imaging, and ultrasound imaging.

In some embodiments, the exosome mimetic is visualized via MRI.

In some embodiments, the exosome mimetic further comprises a diagnostic agent. In some embodiments, the diagnostic agent is a targeting moiety. In some embodiments, the targeting moiety targets a biomarker of cancer. In some embodiments, the cancer is breast cancer. In some embodiments, the biomarker is ICAM1 or HER2.

### BRIEF DESCRIPTION OF THE DRAWINGS

The accompanying drawings are not intended to be drawn to scale. In the drawings, each identical or nearly identical component that is illustrated in various FIGs. is represented by a like numeral. For purposes of clarity, not every component may be labeled in every drawing.

In the drawings:

FIGS. 1A-1I show the characterizations of IONP-EMs derived from MDA-MB-231 cells by the magnetic extrusion method. FIG. 1A shows representative TEM images of IONPs that were internalized by MDA-MB-231 cells. FIG. 1B shows IONPs encapsulated in the endosomes. FIG. 1C shows an IONP-encapsulated endosome after purification and magnetic separation. FIG. 1D shows the purified IONP-encapsulated endosome extruded into engineered IONP-EM. The arrows indicate the encapsulated IONPs. FIGS. 1E and 1F show hydrodynamic size and immunoblot of Alix protein expression of IONP-EMs and native exosomes, respectively. FIG. 1G shows IONP-EM and total EM yields. FIG. 1H shows protein concentration and hydrodynamic size of IONP-EMs in five independent repeats. FIG. 11 shows IONP-EMs increased MDA-MB-231 cell proliferation.

FIG. 2 shows doxorubicin encapsulation efficiency of EMs. Doxorubicin was loaded into mouse fibroblast 3T3 cell-derived EMs using direct encapsulation (left) and ammonium sulfate gradient loading (right) methods.

FIG. 3 shows anti-cancer activity of Dox-EMs in treating human breast cancer MDA-MB-231 cells.

FIG. 4 shows anti-cancer activity of Dox-EMs in treating human breast cancer MDA-MB-436 cells.

### DETAILED DESCRIPTION OF CERTAIN EMBODIMENTS

Production of extracellular vesicles (e.g., exosomes) in large scale is very expensive, time-consuming and labor-intensive. Moreover, the variability between different preparations of exosomes is high and the resulting preparations are often not appropriate for clinical applications. Further, exosomes produced via extrusion of cell plasma membrane in known methods, though similar to native exosomes in size, have different composition and biological function from native exosomes.

The magnetic extrusion methods described herein yield endosome-derived nanoscale vesicles (termed herein “exosome mimetics (EMs)”) that are homogenous in structure and function and retain the biological function of native exosomes. EMs that are endosome-derived have a unique composition enriched in endosomal proteins, more akin to native exosomes in composition and biological function. As shown herein, the methods are suitable for consistent and large scale production of EMs that retain the biological property of exosomes, making these EMs suitable as delivery tools for agents (e.g., therapeutic or diagnostic agents). Further, agents can be loaded to the EMs during production with high encapsulation efficiency.

Accordingly, some aspects of the present disclosure provide methods of producing an exosome mimetic, the method comprising: (i) incubating a cell with a magnetic nanoparticle such that the magnetic nanoparticle enters an endosome in the cell; (ii) lysing the cell to produce a cell lysate containing the endosome; (iii) isolating the endosome encapsulating the magnetic nanoparticle from the cell lysate in step (ii); and (iv) extruding the isolated endosome obtained in step (iii) through a nanoporous membrane to produce the exosome mimetic.

An “exosome” is a small cell-derived vesicle and is of endocytic origin. Exosomes are vehicles for the removal of unnecessary cellular proteins and are considered as important drivers of intercellular communication. Exosomes are found in all biofluids including blood, milk, urine, sweat, tears, and culture supernatant.

During the biogenesis of exosomes, early endosomes loaded with ubiquitinated proteins, upon recognition by ESCRT (Endosomal Sorting Complex Required for Transport), allow the formation of intraluminal vesicles (ILVs), which in turn become multivesicular bodies (MVBs), some of which are degraded in lysosomes. The fusion of MVBs with the plasma membrane causes the release of exosomes into the extracellular space.

Exosomes contain a complex composition of molecules, including proteins, lipids, microRNA, and mRNA, which are cataloged in the EXoCarta database (exocarta.org). The most common exosomal proteins are membrane transporters and fusion proteins (Annexins, GTPases and flotillin), heat shock proteins, tetraspanins (CD9, CD63 and CD81), MVB synthesis proteins (Alix and TSG101), lipid-related proteins and phospholipases. Proteins such as CD9, CD63, CD81, TSG101, Alix and HSP70 are common to most exosomes. Exosomes are enriched with lipids like cholesterol, sphingolipids, ceramide, glycolipid GM3, and glycerophospholipids containing long, saturated fatty-acyl chains.

Exosomes play a key role in cell-to-cell communication by merging with a recipient cell. Exosomes may remain stably associated with the plasma membrane or are internalized via an endocytic pathway, releasing their contents. The biological property of the target cell can then be altered at the genetic level (exosomal RNA), epigenetic level (exosomal miRNA) or at the protein level. Beneficial (e.g. enhancing the immune status) or detrimental (e.g. disseminating pathogenesis) outcomes are possible with these interactions.

An “exosome mimetic,” as used herein, refers to a nano-scale membranous vesicle originated from the endosomal system of a cell. The exosome mimetic of the present disclosure are akin to native exosomes in its structure and biological functions. For example, the exosome mimetic of the present disclosure is a vesicle comprising a lipid bilayer. In some embodiments, the exosome mimetic has one or more known biomarkers of a native exosome, e.g., without limitation, Alix, TSG101, CD9, CD63 and CD81, and HSP70. Cells from which the exosome mimetics can be produced from include, without limitation: bone marrow derived cells, immune cells, red blood cells, epithelial cells, stem cells, and endothelial cells.

A “magnetic nanoparticle” refers to a nanoparticle that can be manipulated using magnetic fields. Such particles commonly consist of two components, a magnetic material, often iron, nickel and cobalt, and a chemical component that has functionality. Magnetic nanoparticles can be iron-based, cobalt-based, nickel-based, or manganese-based (e.g., as described in Kudr et al. (Nanomaterials (Basel). 2017 September; 7(9): 243; incorporated herein by reference).

Non-limiting examples of magnetic nanoparticles that may be used in accordance with the present disclosure include: ferrite nanoparticles (also termed iron oxide nanoparticles), ferrites nanoparticles with a shell, metallic nanoparticles, and metallic nanoparticles with a shell.

Ferrite nanoparticles or iron oxide nanoparticles (iron oxides in crystal structure of maghemite or magnetite) are the most explored magnetic nanoparticles to date. Once the ferrite particles become smaller than 128 nm they become superparamagnetic which prevents self-agglomeration since they exhibit their magnetic behavior only when an external magnetic field is applied. The magnetic moment of ferrite nanoparticles can be greatly increased by controlled clustering of a number of individual superparamagnetic nanoparticles into superparamagnetic nanoparticle clusters, namely magnetic nanobeads. With the external magnetic field switched off, the remanence falls back to zero. Just like non-magnetic oxide nanoparticles, the surface of ferrite nanoparticles is often modified by surfactants, silica, silicones or phosphoric acid derivatives to increase their stability in solution.

The surface of a maghemite or magnetite magnetic nanoparticle is relatively inert and does not usually allow strong covalent bonds with functionalization molecules. However, the reactivity of the magnetic nanoparticles can be improved by coating a layer of silica onto their surface. The silica shell can be easily modified with various surface functional groups via covalent bonds between organo-silane molecules and silica shell. In addition, some fluorescent dye molecules can be covalently bonded to the functionalized silica shell (e.g., ferrites nanoparticles with shell).

Metallic nanoparticles can be made smaller than their oxide counterparts and may be beneficial for some technical applications. Metallic nanoparticles are pyrophoric and reactive to oxidizing agents to various degrees. The metallic core of magnetic nanoparticles may be passivated by gentle oxidation, surfactants, polymers and precious metals. In an oxygen environment, Co nanoparticles form an anti-ferromagnetic CoO layer on the surface of the Co nanoparticle (e.g., metallic nanoparticles with shell). Nanoparticles with a magnetic core consisting either of elementary Iron or Cobalt with a nonreactive shell made of graphene have been synthesized.

In some embodiments, the magnetic nanoparticle used in the methods described herein is an iron oxide nanoparticle (IONP). An “iron oxide nanoparticle (IONP)” typically have diameters between about 1 and 100 nanometers. The two main forms of IONP are magnetite

(Fe3O4) and its oxidized form maghemite (γ-Fe2O3). Magnetite has an inverse spinel structure with oxygen forming a face-centered cubic crystal system. In magnetite, all tetrahedral sites are occupied by Fe3+ and octahedral sites are occupied by both Fe3+ and Fe2+. Maghemite differs from magnetite in that all or most of the iron is in the trivalent state (Fe3+) and by the presence of cation vacancies in the octahedral sites. Maghemite has a cubic unit cell in which the cations are distributed randomly over the 8 tetrahedral and 16 octahedral sites (e.g., as described in Laurent et al., Chemical Reviews. 108 (6): 2064-110, incorporated herein by reference). IONPs (e.g., magnetite and maghemite) are biocompatible and potentially non-toxic to humans. Iron oxide is easily degradable and therefore useful for in vivo applications.

To produce the exosome mimetic, cells are incubated with magnetic nanoparticles (e.g., IONPs) for a period of time. The incubation may be under conditions suitable for the maintenance and/or growth of the cells used. One skilled in the art is able to determine the conditions such as temperature, duration, and/or media for incubation. In some embodiments, the cells are incubated with magnetic nanoparticles (e.g., IONPs) at 25° C.-37° C. (e.g., 25, 26, 27, 28, 29, 30, 31, 32, 33, 34, 35, 36, 37, 38, 39, or 40° C.). In some embodiments, the cells are incubated with magnetic nanoparticles (e.g., IONPs) at 37° C. In some embodiments, the cells are incubated with magnetic nanoparticles (e.g., IONPs) for 1-24 hours (e.g., 1, 2, 3, 4, 5, 6, 7, 8, 9, 10, 11, 12, 13, 14, 15, 16, 17, 18, 19, 20, 21, 22, 23, 24 hours. In some embodiments, the cells are incubated with magnetic nanoparticles (e.g., IONPs) for less than an hour. In some embodiments, the cells are incubated with magnetic nanoparticles (e.g., IONPs) for more than 24 hours.

In some embodiments, the magnetic nanoparticles enter the cells via endocytosis. “Endocytosis” is a cellular process in which substances are brought into the cell. The material to be internalized is surrounded by an area of cell membrane, which then buds off inside the cell to form a vesicle containing the ingested material. Endocytosis is a form of active transport.

Magnetic nanoparticles (e.g., IONPs) that enter the cells via endocytosis are included in endosomes. An “endosome” refers to a membrane-bound compartment inside eukaryotic cells. It is a compartment of the endocytic membrane transport pathway originating from the trans Golgi membrane. Endosomes can be categorized into early endosomes, recycling endosomes, and late endosomes. Early endosomes are the first compartment of the endocytic pathway. Early endosomes are often located in the periphery of the cell, and receive most types of vesicles coming from the cell surface. Early endosomes have a characteristic tubulo-vesicular structure and are principally sorting organelles where many endocytosed ligands dissociate from their receptors in the acid pH of the compartment, and from which many of the receptors recycle to the cell surface (via tubules). Early endosomes are also the sites of sorting into transcytotic pathway to later compartments (like late endosomes or lysosomes). Recycling endosome are often considered as a sub-compartment of the early endosome that recycles internalized cargoes to the plasma membrane. Late endosomes receive endocytosed material en route to lysosomes, usually from early endosomes in the endocytic pathway, from trans-Golgi network (TGN) in the biosynthetic pathway, and from phagosomes in the phagocytic pathway. Late endosomes often contain proteins characteristic of nucleosomes, mitochondria and mRNAs including lysosomal membrane glycoproteins and acid hydrolases. Late endosomes are acidic (about pH 5.5), and are part of the trafficking pathway of mannose-6-phosphate receptors. Late endosomes are thought to mediate a final set of sorting events prior the delivery of material to lysosomes.

After incubation is completed, the cells are lysed. “Lyse” a cell means to disrupt the plasma membrane of a cell such that the contents of the cell are released. Any method suitable for lysing a cell may be used, e.g., mechanical disruption, liquid homogenization, high frequency sound waves, freeze/thaw cycles, sonication or manual grinding. In some embodiments, the cells are lysed via homogenization.

After homogenization, the endosomes that contain the magnetic nanoparticles (e.g., IONPs) are separated from the cell lysates. In some embodiments, the endosomes that contain the magnetic nanoparticles (e.g., IONPs) are separated using a magnetic separator. A magnetic separator can exert a magnetic force which extracts magnetically susceptible materials (e.g., endosomes that contain the magnetic nanoparticles) from the cell lysates. The remaining cell lysates after the endosomes that contain the magnetic nanoparticles (e.g., IONPs) are extracted may be discarded. In some embodiments, the separated endosomes that contain the magnetic nanoparticles (e.g., IONPs) are subjected to several steps of washing to remove any impurities (e.g., proteins, nucleic acids or other materials that typically exist in cell lysates).

The separated endosomes are then extruded through a nanoporous membrane to produce the EMs. A “nanoporous membrane” is a membrane containing regular organic or inorganic framework supporting a regular, porous structure. In some embodiments, the nanoporous membrane is a track-etched polycarbonate (PCTE) nanoporous membrane. In some embodiments, the pores of the nanoporous membrane are 20-400 nm (e.g., 20, 50, 100, 150, 200, 250, 300, 350, or 400 nm). In some embodiments, the pores of the nanoporous membrane are 100 nm. Nanoporous membranes (e.g., track-etched polycarbonate (PCTE) nanoporous membrane) are commercially available, e.g., from SterliTech corporation (WA, USA). In some embodiments, the extrusion step is carried out using a Lipex™ extruder, which is commercially available, e.g., from Transferra Nanosciences Inc. (Canada).

In some embodiments, the methods described herein further comprise removing unencapsulated magnetic nanoparticles (e.g., IONPs) from the resulting EMs after the extrusion step. In some embodiments, the unencapsulated magnetic nanoparticles (e.g., IONPs) are removed from the EMs via size exclusion chromatography. “Size exclusion chromatography (SEC),” also known as gel filtration, separates molecules by differences in size as they pass through a SEC resin packed in a column. During SEC, molecules do not bind to the chromatography resin. SEC resins consist of a porous matrix of spherical particles that lack reactivity and adsorptive properties. After a sample has been applied, molecules larger than the pores are unable to diffuse into the beads, so they elute first. Molecules that range in size between the very big and very small can penetrate the pores to varying degrees based on their size. If a molecule is smaller than the smallest of the pores in the resin, it will be able to enter the total pore volume. Molecules that enter the total pore volume are eluted last. The unencapsulated magnetic nanoparticles (e.g., IONPs) have smaller size than the EMs, which can be separated by SEC.

In some embodiments, the methods described herein further comprise isolating EMs encapsulating magnetic nanoparticles (e.g., IONPs) from empty EMs. In some embodiments, this step is carried out using a magnetic separator.

In some embodiments, the resulting isolated EMs encapsulating magnetic nanoparticles (e.g., IONPs) may be further processed to remove the magnetic nanoparticles (e.g., IONPs) from the EM.

In some embodiments, the EM produced using the methods described herein is 20-400 nm in diameter. For example, the EM produced using the methods described herein may be 20-400, 20-350, 20-300, 20-250, 20-200, 20-150, 20-100, 20-50, 50-400, 50-350, 50-300, 50-250, 50-200, 50-150, 50-100, 100-400, 100-350, 100-300, 100-250, 100-200, 100-150, 150-400, 150-350, 150-300, 150-250, 150-200, 200-400, 200-350, 200-300, 200-250, 250-400, 250-350, 250-300, 300-400, 300-350, or 350-400 nm in diameter. In some embodiments, the EM produced using the methods described herein is 20, 50, 100, 150, 200, 250, 300, 350, or 400 nm in diameter. In some embodiments, the EM produced using the methods described herein is 100 nm in diameter.

In some embodiments, the EMs produced using the methods described herein retain the composition (e.g., biomarkers) and/or biological functions of a natural exosome. For example, in some embodiments, the EMs produced using the methods described herein comprises a known exosome marker (e.g., Alix). In some embodiments, the EMs produced using the methods described herein comprises a known exosome marker (e.g., Alix) at a level comparable (e.g., with less than 20%, less than 15%, less than 10%, less than 5%, or less than 1% difference) that of a native exosome.

In some embodiments, the methods described herein can be used to produce EMs having an encapsulated agent (e.g., a therapeutic agent or a diagnostic agent). For example, the magnetic nanoparticle (e.g., an IONP) can be conjugated to an agent (e.g., a therapeutic agent or a diagnostic agent). A “therapeutic agent” refers to an agent that has therapeutic effects to a disease or disorder. A therapeutic agent may be, without limitation, proteins, peptides, nucleic acids, polysaccharides and carbohydrates, lipids, glycoproteins, small molecules, gene editing agents (e.g., CRISPR/Cas9 systems, ZNF, or TALEN) or synthetic organic and inorganic drugs. In some embodiments, the therapeutic agent is an anti-inflammatory agent, a vaccine antigen, a vaccine adjuvant, an antibody, a ScFv, a nanobody, and enzyme, an anti-cancer drug or chemotherapeutic drug, a clotting factor, a hormone, a steroid, a cytokine, an antibiotic, or a drug for the treatment of a cardiovascular disease, a lung disease, a renal disease, an infectious disease, an autoimmune disease, an immune deficiency, allergy, a blood disorder, a metabolic disorder, a skin disease, an eye disease, a brain disease, a respiratory disease, an endocrine system disease, or cancer.

In some embodiments, the therapeutic agent is a vaccine antigen. A “vaccine antigen” is a molecule or moiety that, when administered to a subject, activates or increases the production of antibodies that specifically bind the antigen. In some embodiments, an antigen is a protein or a polysaccharide. Antigens of pathogens are well known to those of skill in the art and include, but are not limited to parts (coats, capsules, cell walls, flagella, fimbriae, and toxins) of bacteria, viruses, and other microorganisms. A vaccine typically comprises an antigen, and is intentionally administered to a subject to induce an immune response in the recipient subject. The antigen may be from a pathogenic virus, bacteria, or fungi.

Examples of pathogenic virus include, without limitation: Retroviridae (e.g., human immunodeficiency viruses, such as HIV-1 (also referred to as HTLV-III, LAV or HTLV-III/LAV, or HIV-III; and other isolates, such as HIV-LP; Picornaviridae (e.g., polio viruses, hepatitis A virus; enteroviruses, human coxsackie viruses, rhinoviruses, echoviruses); Calciviridae (e.g., strains that cause gastroenteritis); Togaviridae (e.g., equine encephalitis viruses, rubella viruses); Flaviridae (e.g., dengue viruses, encephalitis viruses, yellow fever viruses); Coronaviridae (e.g., coronaviruses); Rhabdoviridae (e.g., vesicular stomatitis viruses, rabies viruses); Filoviridae (e.g., ebola viruses); Paramyxoviridae (e.g., parainfluenza viruses, mumps virus, measles virus, respiratory syncytial virus); Orthomyxoviridae (e.g., influenza viruses); Bungaviridae (e.g., Hantaan viruses, bunga viruses, phleboviruses and Nairo viruses); Arena viridae (hemorrhagic fever viruses); Reoviridae (e.g., reoviruses, orbiviurses and rotaviruses); Birnaviridae; Hepadnaviridae (Hepatitis B virus); Parvoviridae (parvoviruses); Papovaviridae (papilloma viruses, polyoma viruses); Adenoviridae (most adenoviruses); Herpesviridae (herpes simplex virus (HSV) 1 and 2, varicella zoster virus, cytomegalovirus (CMV), herpes viruses); Poxviridae (variola viruses, vaccinia viruses, pox viruses); and Iridoviridae (e.g., African swine fever virus); and unclassified viruses (e.g., the etiological agents of Spongiform encephalopathies, the agent of delta hepatitis (thought to be a defective satellite of hepatitis B virus), the agents of non-A, non-B hepatitis (class 1=internally transmitted; class 2=parenterally transmitted (i.e., Hepatitis C); Norwalk and related viruses, and astroviruses).

Examples of pathogenic bacteria include, without limitation: Helicobacter pyloris, Borelia burgdorferi, Legionella pneumophilia, Mycobacteria spp. (e.g., M. tuberculosis, M. avium, M. intracellulare, M. kansasii, M. gordonae), Staphylococcus aureus, Neisseria gonorrhoeae, Neisseria meningitidis, Listeria monocytogenes, Streptococcus pyogenes (Group A Streptococcus), Streptococcus agalactiae (Group B Streptococcus), Streptococcus (viridans group), Streptococcus faecalis, Streptococcus bovis, Streptococcus (anaerobic spp.), Streptococcus pneumoniae, pathogenic Campylobacter sp., Enterococcus sp., Haemophilus influenzae, Bacillus anthracis, Corynebacterium diphtheriae, Corynebacterium sp., Erysipelothrix rhusiopathiae, Clostridium perfringens, Clostridium tetani, Enterobacter aerogenes, Klebsiella pneumoniae, Pasturella multocida, Bacteroides sp., Fusobacterium nucleatum, Streptobacillus moniliformis, Treponema pallidum, Treponema pertenue, Leptospira, and Actinomyces israelli.

Examples of pathogenic fungi include, without limitation: Cryptococcus neoformans, Histoplasma capsulatum, Coccidioides immitis, Blastomyces dermatitidis, Chlamydia trachomatis, Candida albicans. Other infectious organisms (i.e., protists) include: Plasmodium falciparum and Toxoplasma gondii.

In some embodiments, the therapeutic agent is an agent that induces immunological tolerance. Immunologic tolerance is a state of immune unresponsiveness specific to a particular antigen or set of antigens induced by previous exposure to that antigen or set. In some embodiments, the immunologic tolerance is oral tolerance. Oral tolerance is the state of local and systemic immune unresponsiveness that is induced by oral administration of innocuous antigen such as food proteins. In some embodiments, the therapeutic agent is an agent for induce immunological tolerance for the treatment of allergy or autoimmune disease (e.g., multiple sclerosis).

Other non-limiting examples of agents that may be conjugated to the magnetic nanoparticles (e.g., IONPs) and encapsulated in the EMs produced using the methods described herein are provided.

Non-limiting, exemplary chemopharmaceutically compositions include, Actinomycin, All-trans retinoic acid, Azacitidine, Azathioprine, Bleomycin, Bortezomib, Carboplatin, Capecitabine, Cisplatin, Chlorambucil, Cyclophosphamide, Cytarabine, Daunorubicin, Docetaxel, Doxifluridine, Doxorubicin, Epirubicin, Epothilone, Etoposide, Fluorouracil, Gemcitabine, Hydroxyurea, Idarubicin, Imatinib, Irinotecan, Mechlorethamine, Mercaptopurine, Methotrexate, Mitoxantrone, Oxaliplatin, Paclitaxel, Pemetrexed, Teniposide, Tioguanine, Topotecan, Valrubicin, Vinblastine, Vincristine, Vindesine, and Vinorelbine.

Examples of antineoplastic compounds include, without limitation: nitrosoureas, e.g., carmustine, lomustine, semustine, strepzotocin; Methylhydrazines, e.g., procarbazine, dacarbazine; steroid hormones, e.g., glucocorticoids, estrogens, progestins, androgens, tetrahydrodesoxycaricosterone, cytokines and growth factors; Asparaginase.

Examples of immunoactive compounds include, without limitation: immunosuppressives, e.g., pyrimethamine, trimethopterin, penicillamine, cyclosporine, azathioprine; immunostimulants, e.g., levamisole, diethyl dithiocarbamate, enkephalins, endorphins.

Examples of antimicrobial compounds include, without limitation: antibiotics, e.g., beta lactam, penicillin, cephalosporins, carbapenims and monobactams, beta-lactamase inhibitors, aminoglycosides, macrolides, tetracyclins, spectinomycin; antimalarials, amebicides, antiprotazoal, antifungals, e.g., amphotericin beta or clotrimazole, antiviral, e.g., acyclovir, idoxuridine, ribavirin, trifluridine, vidarbine, gancyclovir.

Examples of parasiticides include, without limitation: antihalmintics, radiopharmaceutics, gastrointestinal drugs.

Examples of hematologic compounds include, without limitation: immunoglobulins; blood clotting proteins; e.g., antihemophilic factor, factor IX complex; anticoagulants, e.g., dicumarol, heparin Na; fibrolysin inhibitors, tranexamic acid.

Examples of cardiovascular drugs include, without limitation: peripheral antiadrenergic drugs, centrally acting antihypertensive drugs, e.g., methyldopa, methyldopa HCl; antihypertensive direct vasodilators, e.g., diazoxide, hydralazine HCl; drugs affecting renin-angiotensin system; peripheral vasodilators, phentolamine; antianginal drugs; cardiac glycosides; inodilators; e.g., amrinone, milrinone, enoximone, fenoximone, imazodan, sulmazole; antidysrhythmic; calcium entry blockers; drugs affecting blood lipids; ranitidine, bosentan, rezulin.

Examples of respiratory drugs include, without limitation: sypathomimetic drugs: albuterol, bitolterol mesylate, dobutamine HCl, dopamine HCl, ephedrine SO, epinephrine, fenfluramine HCl, isoproterenol HCl, methoxamine HCl, norepinephrine bitartrate, phenylephrine HCl, ritodrine HCl; cholinomimetic drugs, e.g., acetylcholine Cl; anticholinesterases, e.g., edrophonium Cl; cholinesterase reactivators; adrenergic blocking drugs, e.g., acebutolol HCl, atenolol, esmolol HCl, labetalol HCl, metoprolol, nadolol, phentolamine mesylate, propanolol HCl; antimuscarinic drugs, e.g., anisotropine methylbromide, atropine SO4, clinidium Br, glycopyrrolate, ipratropium Br, scopolamine HBr.

Examples of neuromuscular blocking drugs include, without limitation: depolarizing, e.g., atracurium besylate, hexafluorenium Br, metocurine iodide, succinylcholine Cl, tubocurarine Cl, vecuronium Br; centrally acting muscle relaxants, e.g., baclofen.

Examples of neurotransmitters and neurotransmitter agents include, without limiation: acetylcholine, adenosine, adenosine triphosphate, amino acid neurotransmitters, e.g., excitatory amino acids, GABA, glycine; biogenic amine neurotransmitters, e.g., dopamine, epinephrine, histamine, norepinephrine, octopamine, serotonin, tyramine; neuropeptides, nitric oxide, K+channel toxins,

Examples of antiparkinson drugs include, without limiation: amaltidine HCl, benztropine mesylate, e.g., carbidopa.

Examples of diuretic drugs include, without limitation: dichlorphenamide, methazolamide, bendroflumethiazide, polythiazide.

Examples of uterine, antimigraine drugs include, without limitation: carboprost tromethamine, mesylate, methysergide maleate.

Examples of hormones include, without limitation: pituitary hormones, e.g., chorionic gonadotropin, cosyntropin, menotropins, somatotropin, iorticotropin, protirelin, thyrotropin, vasopressin, lypressin; adrenal hormones, e.g., beclomethasone dipropionate, betamethasone, dexamethasone, triamcinolone; pancreatic hormones, e.g., glucagon, insulin; parathyroid hormone, e.g., dihydrochysterol; thyroid hormones, e.g., calcitonin etidronate disodium, levothyroxine Na, liothyronine Na, liotrix, thyroglobulin, teriparatide acetate; antithyroid drugs; estrogenic hormones; progestins and antagonists, hormonal contraceptives, testicular hormones; gastrointestinal hormones: cholecystokinin, enteroglycan, galanin, gastric inhibitory polypeptide, epidermal growth factor-urogastrone, gastric inhibitory polypeptide, gastrin-releasing peptide, gastrins, pentagastrin, tetragastrin, motilin, peptide YY, secretin, vasoactive intestinal peptide, sincalide.

Examples of enzymes include, without limitation: lysosomal storage enzymes, hyaluronidase, streptokinase, tissue plasminogen activator, urokinase, PGE-adenosine deaminase, oxidoreductases, transferases, polymerases, hydrolases, lyases, synthases, isomerases, and ligases, digestive enzymes (e.g., proteases, lipases, carbohydrases, and nucleases). In some embodiments, the enzyme is selected from the group consisting of lactase, beta-galactosidase, a pancreatic enzyme, an oil-degrading enzyme, mucinase, cellulase, isomaltase, alginase, digestive lipases (e.g., lingual lipase, pancreatic lipase, phospholipase), amylases, cellulases, lysozyme, proteases (e.g., pepsin, trypsin, chymotrypsin, carboxypeptidase, elastase,), esterases (e.g. sterol esterase), disaccharidases (e.g., sucrase, lactase, beta-galactosidase, maltase, isomaltase), DNases, and RNases.

Examples of intravenous anesthetics include, without limitation: droperidol, etomidate, fetanyl citrate/droperidol, hexobarbital, ketamine HCl, methohexital Na, thiamylal Na, thiopental Na.

Examples of antiepileptics include, without limitation, carbamazepine, clonazepam, divalproex Na, ethosuximide, mephenytoin, paramethadione, phenytoin, primidone.

Examples of peptides and proteins that may be used as therapeutic agents include, without limiation: ankyrins, arrestins, bacterial membrane proteins, clathrin, connexins, dystrophin, endothelin receptor, spectrin, selectin, cytokines; chemokines; growth factors, insulin, erythropoietin (EPO), tumor necrosis factor (TNF), neuropeptides, neuropeptide Y, neurotensin, transforming growth factor alpha, transforming growth factor beta, interferon (IFN), and hormones, growth inhibitors, e.g., genistein, steroids etc; glycoproteins, e.g., ABC transporters, platelet glycoproteins, GPIb-IX complex, GPIIb-IIIa complex, vitronectin, thrombomodulin, CD4, CD55, CD58, CD59, CD44, lymphocye function-associated antigen, intercellular adhesion molecule, vascular cell adhesion molecule, Thy-1, antiporters, CA-15-3 antigen, fibronectins, laminin, myelin-associated glycoprotein, GAP, GAP-43, Exendin-4, and GLP-1.

Examples of cytokines and cytokine receptors include, without limitation: interleukin-1 (IL-1), IL-2, IL-3, IL-4, IL-5, IL-6, IL-7, IL-8, IL-9, IL-10, IL-11, IL-12, IL-13, IL-14, IL-15, IL-16, IL-17, IL-18, IL-1 receptor, IL-2 receptor, IL-3 receptor, IL-4 receptor, IL-5 receptor, IL-6 receptor, IL-7 receptor, IL-8 receptor, IL-9 receptor, IL-10 receptor, IL-11 receptor, IL-12 receptor, IL-13 receptor, IL-14 receptor, IL-15 receptor, IL-16 receptor, IL-17 receptor, IL-18 receptor, lymphokine inhibitory factor, macrophage colony stimulating factor, platelet derived growth factor, stem cell factor, tumor growth factor beta, tumor necrosis factor, lymphotoxin, Fas, granulocyte colony stimulating factor, granulocyte macrophage colony stimulating factor, interferon-alpha, interferon-beta, interferon-gamma.

Examples of growth factors and protein hormones include, without limitation: erythropoietin, angiogenin, hepatocyte growth factor, fibroblast growth factor, keratinocyte growth factor, nerve growth factor, tumor growth factor-alpha, thrombopoietin, thyroid stimulating factor, thyroid releasing hormone, neurotrophin, epidermal growth factor, VEGF, ciliary neurotrophic factor, LDL, somatomedin, insulin growth factor, insulin-like growth factor I and II.

Examples of chemokines include, without limitation: ENA-78, ELC, GRO-alpha, GRO-beta, GRO-gamma, HRG, LIF, IP-10, MCP-1, MCP-2, MCP-3, MCP-4, MIP-lalpha, MIP-1beta, MIG, MDC, NT-3, NT-4, SCF, LIF, leptin, RANTES, lymphotactin, eotaxin-1, eotaxin-2, TARC, TECK, WAP-1, WAP-2, GCP-1, GCP-2; alpha-chemokine receptors: CXCR1, CXCR2, CXCR3, CXCR4, CXCR5, CXCR6, CXCR7; beta-chemokine receptors: CCR1, CCR2, CCR3, CCR4, CCR5, CCR6, CCR7.

In some embodiments, antibodies that may be used as the therapeutic agents in accordance with the present disclosure include, without limitation: (a) anti-cluster of differentiation antigen CD-1 through CD-166 and the ligands or counter receptors for these molecules; (b) anti-cytokine antibodies, e.g., anti-IL-1 through anti-IL-18 and the receptors for these molecules; (c) anti-immune receptor antibodies, antibodies against T cell receptors, major histocompatibility complexes I and II, B cell receptors, selectin killer inhibitory receptors, killer activating receptors, OX-40, MadCAM-1, Gly-CAM1, integrins, cadherens, sialoadherens, Fas, CTLA-4, Fc.gamma.-receptors, Fcalpha-receptors, Fc.epsilon.-receptors, Fc.mu.-receptors, and their ligands; (d) anti-metalloproteinase antibodies, e.g., collagenase, MMP-1 through MMP-8, TIMP-1, TIMP-2; anti-cell lysis/proinflammatory molecules, e.g., perforin, complement components, prostanoids, nitron oxide, thromboxanes; and (e) anti-adhesion molecules, e.g., carcioembryonic antigens, lamins, fibronectins.

Other non-limiting, exemplary antibodies and fragments thereof include: bevacizumab (AVASTIN®), trastuzumab (HERCEPTIN®), alemtuzumab (CAMPATH®, indicated for B cell chronic lymphocytic leukemia,), gemtuzumab (MYLOTARG®, hP67.6, anti-CD33, indicated for leukemia such as acute myeloid leukemia), rituximab (RITUXAN®), tositumomab (BEXXAR®, anti-CD20, indicated for B cell malignancy), MDX-210 (bispecific antibody that binds simultaneously to HER-2/neu oncogene protein product and type I Fc receptors for immunoglobulin G (IgG) (Fc gamma RI)), oregovomab (OVAREX®, indicated for ovarian cancer), edrecolomab (PANOREX®), daclizumab (ZENAPAX®), palivizumab (SYNAGIS®, indicated for respiratory conditions such as RSV infection), ibritumomab tiuxetan (ZEVALIN®, indicated for Non-Hodgkin's lymphoma), cetuximab (ERBITUX®), MDX-447, MDX-22, MDX-220 (anti-TAG-72), IOR-C5, IOR-T6 (anti-CD1), IOR EGF/R3, celogovab (ONCOSCINT® OV103), epratuzumab (LYMPHOCIDE®), pemtumomab (THERAGYN®) and Gliomab-H (indicated for brain cancer, melanoma). Other antibodies and antibody fragments are contemplated and may be used in accordance with the disclosure. In some embodiments, the therapeutic agent is a nanobody. A “nanobody” is a therapeutic protein based on single-domain antibody fragments that contain the unique structural and functional properties of naturally-occurring heavy chain only antibodies.

In some embodiments, the therapeutic agent is a ligand for a cell receptor (e.g., without limitation, a growth factor receptor, a G-protein coupled receptor, or a toll-like receptor).

A regulatory protein that can be used as a therapeutic agent described herein may be, in some embodiments, a transcription factor or a immunoregulatory protein. Non-limiting, exemplary transcriptional factors include: those of the NFKB family, such as Rel-A, c-Rel, Rel-B, p50 and p52; those of the AP-1 family, such as Fos, FosB, Fra-1, Fra-2, Jun, JunB and JunD; ATF; CREB; STAT-1, -2, -3, -4, -5 and -6; NFAT-1, -2 and -4; MAF; Thyroid Factor; IRF; Oct-1 and -2; NF-Y; Egr-1; and USF-43, EGR1, Sp1, and E2F1.

Examples of antiviral agents include, without limitation: reverse transcriptase inhibitors and nucleoside analogs, e.g. ddl, ddC, 3TC, ddA, AZT; protease inhibitors, e.g., Invirase, ABT-538; inhibitors of in RNA processing, e.g., ribavirin.

Other non-limiting examples of known therapeutics which may be delivered by coupling to a magnetic nanoparticle (e.g., IONP) described herein include:

(a) Capoten, Monopril, Pravachol, Avapro, Plavix, Cefzil, Duricef/Ultracef, Azactam, Videx, Zerit, Maxipime, VePesid, Paraplatin, Platinol, Taxol, UFT, Buspar, Serzone, Stadol NS, Estrace, Glucophage (Bristol-Myers Squibb);

(b) Ceclor, Lorabid, Dynabac, Prozac, Darvon, Permax, Zyprexa, Humalog, Axid, Gemzar, Evista (Eli Lily);

(c) Vasotec/Vaseretic, Mevacor, Zocor, Prinivil/Prinizide, Plendil, Cozaar/Hyzaar, Pepcid, Prilosec, Primaxin, Noroxin, Recombivax HB, Varivax, Timoptic/XE, Trusopt, Proscar, Fosamax, Sinemet, Crixivan, Propecia, Vioxx, Singulair, Maxalt, Ivermectin (Merck & Co.);

(d) Diflucan, Unasyn, Sulperazon, Zithromax, Trovan, Procardia XL, Cardura, Norvasc, Dofetilide, Feldene, Zoloft, Zeldox, Glucotrol XL, Zyrtec, Eletriptan, Viagra, Droloxifene, Aricept, Lipitor (Pfizer);

(e) Vantin, Rescriptor, Vistide, Genotropin, Micronase/Glyn./Glyb., Fragmin, Total Medrol, Xanax/alprazolam, Sermion, Halcion/triazolam, Freedox, Dostinex, Edronax, Mirapex, Pharmorubicin, Adriamycin, Camptosar, Remisar, Depo-Provera, Caverject, Detrusitol, Estring, Healon, Xalatan, Rogaine (Pharmacia & Upjohn);

(f) Lopid, Accrupil, Dilantin, Cognex, Neurontin, Loestrin, Dilzem, Fempatch, Estrostep, Rezulin, Lipitor, Omnicef, FemHRT, Suramin, Clinafloxacin (Warner Lambert).

Non-limiting examples of therapeutic agents for eye diseases include: Anti-infective drugs (e.g., Acyclovir, Chloramphenicol, Ciprofloxacin, Gentamicin, Neomycin, Polymyxin B); Anti-inflammatory drugs (e.g., Betamethasone, Dexamethasone, Emedastine, Nedocromil sodium, Prednisolone, Sodium cromoglicate); Artificial tears (e.g., Carmellose, Hydroxyethylcellulose, Hypromellose, Polyvinyl alcohol); and Mydriatics (e.g., Atropine, cyclopentolate, Phenylephrine).

Further non-limiting examples of therapeutic agents may be found in: Goodman and Gilman's The Pharmacological Basis of Therapeutics. 9th ed. McGraw-Hill 1996, incorporated herein by reference.

A “diagnostic agent” refers to an agent that is used for diagnostic purpose, e.g., by detecting another molecule in a cell or a tissue. In some embodiments, the diagnostic agent is an agent that targets (e.g., binds) a biomarker known to be associated with a disease (e.g., a nucleic acid biomarker, protein biomarker, or a metabolite biomarker) in a subject and produces a detectable signal, which can be used to determine the presence/absence of the biomarker, thus to diagnose a disease. For example, the diagnostic agent may be, without limitation, an antibody or an antisense nucleic acid.

In some embodiments, the diagnostic agent contains a detectable molecule. A detectable molecule refers to a moiety that has at least one element, isotope, or a structural or functional group incorporated that enables detection of a molecule, e.g., a protein or polypeptide, or other entity, to which the diagnostic agent binds. In some embodiments, a detectable molecule falls into any one (or more) of five classes: a) an agent which contains isotopic moieties, which may be radioactive or heavy isotopes, including, but not limited to, 2H, 3H, 13C, 14C, 15N, 18F, 31P, 32P, 35S, 67Ga, 76Br, 99mTc (Tc-99m), 111In, 123I, 125I, 131I, 153Gd, 169Yb, and 186Re; b) an agent which contains an immune moiety, which may be an antibody or antigen, which may be bound to an enzyme (e.g., such as horseradish peroxidase); c) an agent comprising a colored, luminescent, phosphorescent, or fluorescent moiety (e.g., such as the fluorescent label fluoresceinisothiocyanat (FITC); d) an agent which has one or more photo affinity moieties; and e) an agent which is a ligand for one or more known binding partners (e.g., biotin-streptavidin, His -NiTNAFK506-FKBP). In some embodiments, a detectable molecule comprises a radioactive isotope. In some embodiments, a detection agent comprises a fluorescent moiety. In some embodiments, the detectable molecule comprises a dye, e.g., a fluorescent dye, e.g., fluorescein isothiocyanate, Texas red, rhodamine, Cy3, Cy5, Cy5.5, Alexa 647 and derivatives. In some embodiments, the detectable molecule comprises biotin. In some embodiments, the detectable molecule is a fluorescent polypeptide (e.g., GFP or a derivative thereof such as enhanced GFP (EGFP)) or a luciferase (e.g., a firefly, Renilla, or Gaussia luciferase). In some embodiments, a detectable molecule may react with a suitable substrate (e.g., a luciferin) to generate a detectable signal. Non-limiting examples of fluorescent proteins include GFP and derivatives thereof, proteins comprising chromophores that emit light of different colors such as red, yellow, and cyan fluorescent proteins, etc. Exemplary fluorescent proteins include, e.g., Sirius, Azurite, EBFP2, TagBFP, mTurquoise, ECFP, Cerulean, TagCFP, mTFP1, mUkG1, mAG1, AcGFP1, TagGFP2, EGFP, mWasabi, EmGFP, TagYPF, EYFP, Topaz, SYFP2, Venus, Citrine, mKO, mKO2, mOrange, mOrange2, TagRFP, TagRFP-T, mStrawberry, mRuby, mCherry, mRaspberry, mKate2, mPlum, mNeptune, T-Sapphire, mAmetrine, mKeima. See, e.g., Chalfie, M. and Kain, SR (eds.) Green fluorescent protein: properties, applications, and protocols (Methods of biochemical analysis, v. 47, Wiley-Interscience, and Hoboken, N.J., 2006, and/or Chudakov, DM, et al., Physiol Rev. 90(3):1103-63, 2010, incorporated herein by reference, for discussion of GFP and numerous other fluorescent or luminescent proteins. In some embodiments, a detectable molecule comprises a dark quencher, e.g., a substance that absorbs excitation energy from a fluorophore and dissipates the energy as heat.

In some embodiments, the therapeutic agent and or diagnostic agent that can be conjugated to the magnetic nanoparticle (e.g., IONP) and be encapsulated in the EM produced using the methods described herein are for treating or diagnosing a brain disease (e.g., without limitation, brain cancers, neurologic disorders, psychological disorders, cerebrovascular vascular disorders (such as cerebrovascular incident, vascular malformations and anomalies, moyamoya disease, venous angiomas), brain trauma, and brain infection.

In some embodiments, the therapeutic agent is for treating brain cancer (e.g., primary brain cancer and/or metastatic brain cancer). “Primary brain cancer” refers to a cancer that starts in the brain. “Metastatic brain cancer” means cancer that starts from other parts of the body (e.g., breast cancer, prostate cancer, lung cancer, colorectal cancer, skin cancer).

In some embodiments, the therapeutic agent for treating brain cancer is a chemotherapeutic agent. A “chemotherapeutic agent” refers is a chemical agent or drugs that are selectively destructive to malignant cells and tissues. Non-limiting, exemplary chemopharmaceutically compositions that may be used in accordance with the present disclosure include, Neratinib or lapatinib, Actinomycin, All-trans retinoic acid, Azacitidine, Azathioprine, Bleomycin, Bortezomib, Carboplatin, Capecitabine, Cisplatin, Chlorambucil, Cyclophosphamide, Cytarabine, Daunorubicin, Docetaxel, Doxifluridine, Doxorubicin, Epirubicin, Epothilone, Etoposide, Fluorouracil, Gemcitabine, Hydroxyurea, Idarubicin, Imatinib, Irinotecan, Mechlorethamine, Mercaptopurine, Methotrexate, Mitoxantrone, Oxaliplatin, Paclitaxel, Pemetrexed, Teniposide, Tioguanine, Topotecan, Valrubicin, Vinblastine, Vincristine, Vindesine, and Vinorelbine.

In some embodiments, the therapeutic agent for treating brain cancer is an immunotherapeutic agent. An “immunotherapeutic agent” refers to an agent that modulates (e.g., suppresses or activates) the immune response to treat a disease. Immunetheraepeutic agents are known to those skilled in the art, e.g., those listed on www.ncbi.nlm.nih.gov/medgen/2637.

In some embodiments, the immunotherapeutic agent is an immune checkpoint inhibitor. An “immune checkpoint” is a protein in the immune system that either enhances an immune response signal (co-stimulatory molecules) or reduces an immune response signal. Many cancers protect themselves from the immune system by exploiting the inhibitory immune checkpoint proteins to inhibit the T cell signal. Exemplary inhibitory checkpoint proteins include, without limitation, Cytotoxic T-Lymphocyte-Associated protein 4 (CTLA-4), Programmed Death 1 receptor (PD-1), T-cell Immunoglobulin domain and Mucin domain 3 (TIM3), Lymphocyte Activation Gene-3 (LAG3), V-set domain-containing T-cell activation inhibitor 1 (VTVN1 or B7-H4), Cluster of Differentiation 276 (CD276 or B7-H3), B and T Lymphocyte Attenuator (BTLA), Galectin-9 (GALS), Checkpoint kinase 1 (Chk1), Adenosine A2A receptor (A2aR), Indoleamine 2,3-dioxygenase (IDO), Killer-cell Immunoglobulin-like Receptor (KIR), Lymphocyte Activation Gene-3 (LAG3), and V-domain Ig suppressor of T cell activation (VISTA).

Some of these immune checkpoint proteins need their cognate binding partners, or ligands, for their immune inhibitory activity. For example, A2AR is the receptor of adenosine A2A and binding of A2A to A2AR activates a negative immune feedback loop. As another example, PD-1 associates with its two ligands, PD-L1 and PD-L2, to down regulate the immune system by preventing the activation of T-cells. PD-1 promotes the programmed cell death of antigen specific T-cells in lymph nodes and simultaneously reduces programmed cell death of suppressor T cells, thus achieving its immune inhibitory function. As yet another example, CTLA4 is present on the surface of T cells, and when bound to its binding partner CD80 or CD86 on the surface of antigen-present cells (APCs), it transmits an inhibitory signal to T cells, thereby reducing the immune response.

An “immune checkpoint inhibitor” is a molecule that prevents or weakens the activity of an immune checkpoint protein, For example, an immune checkpoint inhibitor may inhibit the binding of the immune checkpoint protein to its cognate binding partner, e.g., PD-1, CTLA-4, or A2aR. In some embodiments, the immune checkpoint inhibitor is a small molecule. In some embodiments, the immune checkpoint inhibitors is a nucleic acid aptamer (e.g., a siRNA targeting any one of the immune checkpoint proteins). In some embodiments, the immune checkpoint inhibitor is a recombinant protein. In some embodiments, the immune checkpoint inhibitor is an antibody. In some embodiments, the antibody comprises an anti-CTLA-4, anti-PD-1, anti-PD-L1, anti-TIM3, anti-LAG3, anti-B7-H3, anti-B7-H4, anti-BTLA, anti-GALS, anti-Chk, anti-A2aR, anti-IDO, anti-KIR, anti-LAG3, anti-VISTA antibody, or a combination of any two or more of the foregoing antibodies. In some embodiments, the immune checkpoint inhibitor is a monoclonal antibody. In some embodiments, the immune checkpoint inhibitor comprises anti-PD1, anti-PD-L1, anti-CTLA-4, or a combination of any two or more of the foregoing antibodies. For example, the anti-PD-1 antibody is pembrolizumab (Keytruda®) or nivolumab (Opdivo®) and the anti-CTLA-4 antibody is ipilimumab (Yervoy®). Thus, in some embodiments, the immune checkpoint inhibitor comprises pembrolizumab, nivolumab, ipilimumab, or any combination of two or more of the foregoing antibodies. The examples described herein are not meant to be limiting and that any immune checkpoint inhibitors known in the art and any combinations thereof may be used in accordance with the present disclosure.

In some embodiments, the therapeutic agent for treating brain cancer is an oligonucleotide (e.g., an siRNA, shRNA, or miRNA targeting an oncogene). An “oncogene” is a gene that in certain circumstances can transform a cell into a tumor cell. An oncogene may be a gene encoding a growth factor or mitogen (e.g., c-Sis), a receptor tyrosine kinase (e.g., EGFR, PDGFR, VEGFR, or HER2/neu), a cytoplasmic tyrosine kinase (e.g., Src family kinases, Syk-ZAP-70 family kinases, or BTK family kinases), a cytoplasmic serine/threonine kinase or their regulatory subunits (e.g., Raf kinase or cyclin-dependent kinase), a regulatory GTPase (e.g., Ras), or a transcription factor (e.g., Myc). In some embodiments, the oligonucleotide targets Lipocalin (Lcn2) (e.g., a Lcn2 siRNA). One skilled in the art is familiar with genes that may be targeted for the treatment of cancer.

In some embodiments, the therapeutic agent is a gene editing agent. A “gene editing agent” refers to an agent that is capable of inserting, deleting, or replacing nucleotide(s) in the genome of a living organism. In some embodiments, a genome editing agent is an engineered nuclease that can create site-specific double-strand breaks (DSBs) at desired locations in the genome. The induced double-strand breaks are repaired through nonhomologous end-joining (NHEJ) or homologous recombination (HR), resulting in targeted mutations (‘edits’). As such, the engineered nucleases suitable for genome-editing may be programmed to target any desired sequence in the genome and are also referred to herein as “programmable nucleases.” Suitable programmable nucleases for genome-editing that may be used in accordance with the present disclosure include, without limitation, meganucleases, zinc finger nucleases (ZFNs), transcription activator-like effector-based nucleases (TALEN), and the CRISPR/Cas system. One skilled in the art is familiar with the programmable nucleases and methods of using them for genome-editing. For example, methods of using ZFNs and TALENs for genome-editing are described in Maeder, et al., Mol. Cell 31 (2): 294-301, 2008; Carroll et al., Genetics Society of America, 188 (4): 773-782, 2011; Miller et al., Nature Biotechnology 25 (7): 778-785, 2007; Christian et al., Genetics 186 (2): 757-61, 2008; Li et al., Nucleic Acids Res 39 (1): 359-372, 2010; and Moscou et al., Science 326 (5959): 1501, 2009, incorporated herein by reference.

In some embodiments, the genome-editing agent is a Clustered regularly interspaced short palindromic repeats (CRISPR)/Cas system (e.g., a Cas9 and a guide RNA). A “CRISPR/Cas system” refers to a prokaryotic adaptive immune system that provides protection against mobile genetic elements (viruses, transposable elements and conjugative plasmids). CRISPR clusters contain spacers, sequences complementary to antecedent mobile elements, and target invading nucleic acids. CRISPR clusters are transcribed and processed into CRISPR RNA (crRNA). In type II CRISPR systems correct processing of pre-crRNA requires a trans-encoded small RNA (tracrRNA), endogenous ribonuclease 3 (mc) and a Cas9 protein. The tracrRNA serves as a guide for ribonuclease 3-aided processing of pre-crRNA. Subsequently, Cas9/crRNA/tracrRNA endonucleolytically cleaves linear or circular dsDNA target complementary to the spacer. The target strand not complementary to crRNA is first cut endonucleolytically, then trimmed 3′-5′ exonucleolytically. In nature, DNA-binding and cleavage typically requires protein and both RNAs. However, single guide RNAs (“sgRNA”, or simply “gNRA”) can be engineered so as to incorporate aspects of both the crRNA and tracrRNA into a single RNA species. See, e.g., Jinek et al., Science 337:816-821(2012), incorporated herein by reference.

The anti-cancer agent for treating brain cancer used in accordance with the present disclosure can be any anti-cancer drug known to those skilled in the art, e.g., the drugs listed on www.cancer.gov/about-cancer/treatment/drugs.

In some embodiments, the therapeutic agent is for treating a neurologic disorder. A “neurologic disorder” refers to any disorder of the nervous system (e.g., central nervous system or peripheral nervous system. Structural, biochemical or electrical abnormalities in the brain, spinal cord or other nerves can result in a range of symptoms. Examples of symptoms include paralysis, muscle weakness, poor coordination, loss of sensation, seizures, confusion, pain and altered levels of consciousness. There are many recognized neurological disorders, including, without limitation, neurodegenerative diseases (e.g., without limitation, Alzheimer's disease, Parkinson's disease, Huntington's disease, dementia, amyotrophic lateral sclerosis (ALS), prion disease, and motor neuron diseases), neurobehavioral diseases, and developmental disorders.

One skilled in the art is familiar with therapeutic agents that treat neurologic disorders. For example, the therapeutic agent for treating a neurologic disorder that may be used in accordance with the present disclosure include, without limitation, dopaminergic agents (e.g., dopamine receptor agonists), cholinesterase inhibitors, antipsychotic drugs, anti-inflammatory agents, and brain stimulants. Any of the known agents for treating neurologic disorders can be used in accordance with the present disclosure.

In some embodiments, the therapeutic agent is for treating a psychological disorder. A “psychological disorder” is also referred to as mental disorders or psychiatric disorder. A psychological disorder is a behavioral or mental pattern that causes significant distress or impairment of personal functioning. Such features may be persistent, relapsing and remitting, or occur as a single episode. Many disorders have been described, with signs and symptoms that vary widely between specific disorders. Non-limiting examples of psychological disorders include, post-traumatic stress disorder (PTSD), depressive disorder, major depressive disorders, post-partum depression, bipolar disorder, acute stress disorder, generalized anxiety disorder, obsessive-compulsive disorder, panic disorders, schizophrenia, and trichotillomania.

One skilled in the art is familiar with therapeutic agents (e.g., psychiatric drug) that treat psychological disorders. Non-limiting examples of psychiatric drugs include anti-depressants, anti-psychotics, mood stabilizers, brain stimulants, and anti-anxiety drugs.

In some embodiments, the therapeutic agent is for treating brain trauma (also termed “traumatic brain injury”). “Brain trauma” refers to a form of acquired brain injury that occurs when a sudden trauma causes damage to the brain. Symptoms of brain trauma can be mild, moderate, or severe, depending on the extent of the damage to the brain. A subject with a mild brain trauma may remain conscious or may experience a loss of consciousness for a few seconds or minutes. Other symptoms of mild brain trauma include headache, confusion, lightheadedness, dizziness, blurred vision or tired eyes, ringing in the ears, bad taste in the mouth, fatigue or lethargy, a change in sleep patterns, behavioral or mood changes, and trouble with memory, concentration, attention, or thinking. A subject with a moderate or severe brain trauma may show these same symptoms, but may also have a headache that gets worse or does not go away, repeated vomiting or nausea, convulsions or seizures, an inability to awaken from sleep, dilation of one or both pupils of the eyes, slurred speech, weakness or numbness in the extremities, loss of coordination, and increased confusion, restlessness, or agitation.

One skilled in the art is familiar with therapeutic agents that treat brain trauma. Non-limiting examples of therapeutic agents that treat brain trauma include anti-inflammatory agents, corticosteroids, and coagulant agents.

Non-limiting examples of dopaminergic agents include apomorphine, bromocriptine, cabergoline, dihydrexidine (LS-186,899), dopamine, fenoldopam, piribedil, lisuride, pergolide, pramipexole, ropinirole, and rotigotine.

Cholinesterase inhibitors (also termed “acetylcholinesterase inhibitors”) are agents that prevent the breakdown of acetylcholine in the body. Cholinesterase inhibitors have been used to treat neurologic disorders (e.g., Alzheimer's disease and dementia). Non-limiting examples of Cholinesterase inhibitors include: organophosphates (e.g., echothiophate, diisopropyl fluorophosphate, cadusafos, chlorpyrifos, cyclosarin, dichlorvos, dimethoate, metrifonate, sarin, soman, tabun, diazinon, malathion, parathion, carbamates), carbamates (e.g., aldicarb, bendiocarb, bufencarb, carbaryl, carbendazim, carbetamide, carbofuran, carbosulfan, chlorbufam, chloropropham, ethiofencarb, formetanate, methiocarb, methomyl, oxamyl, phenmedipham, pinmicarb, pirimicarb, propamocarb, propham, propoxur), onchidal, coumarins, physostigmine, neostigmine, pyridostigmine, ambenonium, demecarium, rivastigmine, phenanthrene derivatives, galantamine, caffeine, rosmarinic acid, alpha-pinene, piperidines, donepezil, tetrahydroaminoacridine (THA), edrophonium, huperzine a, ladostigil, ungeremine, lactucopicrin, acotiamide, hybrid/bitopic ligands, dyflos, echothiophate, and parathion. Cholinesterase inhibitors that are in clinical use include, without limitation: Cognex, Namzaric (Pro), Razadyne ER, Aricept ODT (Pro), Reminyl, Exelon (Pro), Aricept (Pro), and Razadyne (Pro).

Any known anti-psychotic drugs may be used in accordance with the present disclosure. Non-limiting examples of antipsychotic drugs include aripiprazole (Abilify), asenapine (Saphris), cariprazine (Vraylar), clozapine (Clozaril), lurasidone (Latuda), olanzapine (Zyprexa), quetiapine (Seroquel), risperidone (Risperdal), and ziprasidone (Geodon), Fluoxetine, Citalopram, Sertraline, Paroxetine, Escitalopram, Clonazepam, Alprazolam, Lorazepam, Methylphenidate, Amphetamine, Dextroamphetamine, Lisdexamfetamine Dimesylate, typical antipsychotics include:, Chlorpromazine, Haloperidol, Perphenazine, Fluphenazine, Aripiprazole, Paliperidone, Lurasidone, Carbamazepine, Lamotrigine, and Oxcarbazepine.

An anti-inflammatory agent is a substance that reduces inflammation (redness, swelling, and pain) in the body. Any known anti-inflammatory agents may be used in accordance with the present disclosure, e.g., the anti-inflammatory agents as described in Maroon et al., Surg Neurol Int. 2010; 1: 80; and Dinarello et al., Cell 140, 935-950, Mar. 19, 2010, incorporated herein by reference.

Any known brain stimulants may be used in accordance with the present disclosure. Brain stimulants may be divided into three categories, short-acting, intermediate-acting, and long-acting. Non-limiting examples of short-acting brain stimulants include: Amphetamine/dextroamphetamine (Adderall), Dextroamphetamine (Dexedrine, ProCentra, Zenzedi), Dexmethylphenidate (Focalin), and Methylphenidate (Ritalin). Non-limiting examples of intermediate-acting brain stimulants include: Amphetamine sulfate (Evekeo) and Methylphenidate (Ritalin SR, Metadate ER, Methylin ER). Non-limiting examples of long-acting brain stimulants include: Amphetamine (Adzenys XR-ODT, Dyanavel XR), Dexmethylphenidate (Focalin XR), Dextroamphetamine (Adderall XR), Lisdexamfetamine (Vyvanse), Methylphenidate (Concerta, Daytrana, Jornay PM, Metadate CD, Quillivant XR, Quillichew ER, Ritalin LA), and mixed salts of a single-entity amphetamine product (Mydayis).

Any known anti-depressants may be used in accordance with the present disclosure. Non-limiting examples of anti-depressants include citalopram (Celexa), escitalopram (Lexapro), fluoxetine (Prozac, Sarafem, Selfemra, Prozac Weekly), fluvoxamine (Luvox), paroxetine (Paxil, Paxil CR, Pexeva), sertraline (Zoloft), vortioxetine (Trintellix, formerly known as Brintellix), vilazodone (Viibryd), duloxetine (Cymbalta), venlafaxine (Effexor), desvenlafaxine (Pristiq, Khedezla), levomilnacipran (Fetzima), amitriptyline (Elavil and Endep are discontinued brands in the US), amoxapine, clomipramine (Anafranil), desipramine (Norpramin), doxepin (Sinequan and Adapin are discontinued brands in the US), imipramine (Tofranil), nortriptyline (Pamelor; Aventyl is a discontinued brand in the US), protriptyline (Vivactil), trimipramine (Surmontil), mirtazapine (Remeron), bupropion (Wellbutrin), trazodone, (Desyrel), trazodone extended release tablets (Oleptro), vortioxetine (Trintellix, formerly known as Brintellix), and vilazodone (Viibryd).

A mood stabilizer is a psychiatric drug used to treat mood disorders characterized by intense and sustained mood shifts (e.g., as seen in patients with typically bipolar disorder type I or type II, borderline personality disorder (BPD) and schizoaffective disorder). Any known mood stabilizers may be used in accordance with the present disclosure. Non-limiting examples of mood stabilizes include: lithium (lithium carbonate or lithium citrate), Divalproex (valproic acid or valproate), Carbamazepine, Oxcarbazepine (Trileptal), and Lamotrigine.

Any known anti-anxiety drugs may be used in accordance with the present disclosure. Non-limiting examples of anti-anxiety drugs include: benzodiazepines, citalopram (Celexa), escitalopram (Lexapro), fluoxetine (Prozac), fluvoxamine (Luvox), paroxetine (Paxil, Pexeva), sertraline (Zoloft), duloxetine (Cymbalta), venlafaxine (Effexor XR), amitriptyline (Elavil), imipramine (Tofranil), nortriptyline (Pamelor), isocarboxazid (Marplan), phenelzine (Nardil), selegiline (Emsam), and tranylcypromine (Parnate). Exemplary benzodiazepines include, without limitation, alprazolam (Xanax), clonazepam (Klonopin), chlordiazepoxide (Librium), diazepam (Valium), and lorazepam (Ativan).

Any known corticosteroids may be used in accordance with the present disclosure. Non-limiting examples of corticosteroids include: bethamethasone (Celestone), prednisone (Prednisone Intensol), prednisolone (Orapred, Prelone), triamcinolone (Aristospan Intra-Articular, Aristospan Intralesional, Kenalog), methylprednisolone (Medrol, Depo-Medrol, Solu-Medrol), dexamethasone (Dexamethasone Intensol, DexPak 10 Day, DexPak 13 Day, DexPak 6 Day), hydrocortisone (Cortef), cortisone, ethamethasoneb (Celestone), Methylprednisolone (Medrol, Depo-Medrol, Solu-Medrol), and Fludrocortisone (Florinef).

Any known coagulant agents may be used in accordance with the present disclosure. Non-limiting examples of coagulant agents include: antihemorrhagic agents, ziolites, desmopressin, coagulation factor concentrates, prothrombin complex concentrate, cryoprecipitate and fresh frozen plasma, recombinant activated human factor VII, tranexamic acid and aminocaproic acid.

In some embodiments, the therapeutic agent is for treating brain infection. “Brain infection” can be caused by viruses, bacteria, fungi, protozoa, or parasites. Another group of brain disorders, called spongiform encephalopathies, are caused by abnormal proteins called prions. Brain infection often also involve other parts of the central nervous system, including the spinal cord. In some instances, infections can cause inflammation of the brain (encephalitis). Viruses are the most common causes of encephalitis. Infections can also cause inflammation of the layers of tissue (meninges) that cover the brain and spinal cord—called meningitis. Often, bacterial meningitis spreads to the brain itself, causing encephalitis. Similarly, viral infections that cause encephalitis often also cause meningitis. Technically, when both the brain and the meninges are infected, the disorder is called meningoencephalitis. However, infection that affects mainly the meninges is usually called meningitis, and infection that affects mainly the brain is usually called encephalitis. Usually in encephalitis and meningitis, infection is not confined to one area. It may occur throughout the brain or within meninges along the entire length of the spinal cord and over the entire brain.

In some embodiments, the therapeutic agent for treating brain infection is selected from known anti-infective agents, e.g., antibiotics for treating bacterial infection, anti-viral agents for treating viral infection, or anti-fungal agents for treating fungal infection, or anti-parasite agents to treat parasitic infection. In some embodiments, the brain infection is prion disease and the therapeutic agent for treat prion disease is an anti-prion antibody.

Any known antimicrobial compounds may be used in accordance with the present disclosure. Non-limiting examples of antimicrobial compounds include, without limitation: antibiotics (e.g., beta lactam, penicillin, cephalosporins, carbapenims and monobactams, beta-lactamase inhibitors, aminoglycosides, macrolides, tetracyclins, spectinomycin), antimalarials, amebicides, antiprotazoal, antifungals (e.g., amphotericin beta or clotrimazole), antiviral (e.g., acyclovir, idoxuridine, ribavirin, trifluridine, vidarbine, ganciclovir). Examples of parasiticides include, without limitation: antihalmintics, Radiopharmaceutics, gastrointestinal drugs.

In some embodiments, the magnetic nanoparticle (e.g., IONP) is conjugated to a targeting moiety. A “targeting moiety” refers to a molecule that can target the magnetic nanopaticle (e.g., IONP) and/or the EM encapsulating the magnetic nanopaticle (e.g., IONP) to a specific cell (e.g., a cancer cell) or a tissue (e.g., muscle). In some embodiments, the targeting moiety is a molecule that specifically binds a target in a specific cell (e.g., a cancer cell) or a tissue (e.g., muscle). For example, the targeting moiety may be an antibody targeting a cancer specific antigen, or a ligand for a cell surface receptor. In some embodiments, the targeting moiety targets a cancer cell. In some embodiments, the targeting moiety is a ICAM-1 antibody and/or a HER2 antibody.

Methods of conjugating a magnetic nanoparticle (e.g., IONP) with an agent (e.g., therapeutic agent or diagnostic agent) or a targeting moiety are known in the art. The conjugation may be covalent or non-covalent. For example, conjugation methods are provided in Guo et al., Proc Natl Acad Sci U S A. 2014 Oct. 14; 111(41):14710-5; Huang et al., International Journal of Nanomedicine 7 Jul. 2016, 11:3087-3099; Cho et al., Small (Weinheim an der Bergstrasse, Germany) 6 Jan. 2013, 9(11):1964-1973; Chorny et al., FASEB Journal, 2 Apr. 2007, 21(10):2510-2519; and Hryhorowicz et al., Mol Biotechnol. 2019 March; 61(3):173-180, incorporated herein by reference.

It is to be understood that, the encapsulate any one of the agents or targeting moieties described herein into the EMs produced using the methods described herein, the agents or targeting moieties can be conjugated to the magnetic nanoparticle (e.g., IONP) prior to EM production, or be loaded to the EM after its production using unconjugated magnetic nanoparticle (e.g., IONP).

The present disclosure, in some aspects, further provides any one of the EMs produced using the methods described herein and compositions comprising any one of the EMs. In some embodiments, the EM comprises a magnetic nanoparticle (e.g., IONP). In some embodiments, the EM comprises a magnetic nanoparticle (e.g., IONP) conjugated (e.g., covalently or non-covalently) to an agent (e.g., therapeutic agent or diagnostic agent) or a targeting moiety. In some embodiments, the EM is an empty EM (e.g., if the magnetic nanoparticle is removed from the EM after its production). In some embodiments, the empty EM is later loaded with an agent (e.g., therapeutic agent or diagnostic agent).

In some embodiments, the EMs produced using the methods described herein can be used as delivery vehicles to deliver agents (e.g., therapeutic agents or diagnostic agents) to a cell (e.g., an in vitro cultured cell, or a cell in vivo in a subject). In some embodiments, the EMs can be used as delivery vehicles to deliver agents (e.g., therapeutic agents or diagnostic agents) to a subject, e.g., for the treatment or diagnosis of a disease.

In some embodiments, the composition is formulated as a pharmaceutical composition for administration to a subject. In some embodiments, the pharmaceutical composition further comprises a pharmaceutically acceptable carrier. “Pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. A “pharmaceutically acceptable carrier” may be a pharmaceutically acceptable material, composition or vehicle, such as a liquid or solid filler, diluent, excipient, solvent or encapsulating material, involved in carrying or transporting the subject agents from one organ, or portion of the body, to another organ, or portion of the body. Each carrier must be “acceptable” in the sense of being compatible with the other ingredients of the formulation and not injurious to the tissue of the patient (e.g., physiologically compatible, sterile, physiologic pH, etc.). The term “carrier” denotes an organic or inorganic ingredient, natural or synthetic, with which the active ingredient is combined to facilitate the application. The components of the pharmaceutical compositions also are capable of being co-mingled with the molecules of the present disclosure, and with each other, in a manner such that there is no interaction which would substantially impair the desired pharmaceutical efficacy. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C2-C12 alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation.

The pharmaceutical compositions may conveniently be presented in unit dosage form and may be prepared by any of the methods well-known in the art of pharmacy. The term “unit dose” when used in reference to a pharmaceutical composition of the present disclosure refers to physically discrete units suitable as unitary dosage for the subject, each unit containing a predetermined quantity of active material calculated to produce the desired therapeutic effect in association with the required diluent; i.e., carrier, or vehicle.

The formulation of the pharmaceutical composition may dependent upon the route of administration. Injectable preparations suitable for parenteral administration or intratumoral, peritumoral, intralesional or perilesional administration include, for example, sterile injectable aqueous or oleaginous suspensions and may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3 propanediol or 1,3 butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil may be employed including synthetic mono- or di-glycerides. In addition, fatty acids such as oleic acid find use in the preparation of injectables. The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

Compositions suitable for oral administration may be presented as discrete units, such as capsules, tablets, lozenges, each containing a predetermined amount of the anti-inflammatory agent. Other compositions include suspensions in aqueous liquids or non-aqueous liquids such as a syrup, elixir or an emulsion.

In some embodiments, the pharmaceutical compositions used for therapeutic administration must be sterile. Sterility is readily accomplished by filtration through sterile filtration membranes (e.g., 0.2 micron membranes). Alternatively, preservatives can be used to prevent the growth or action of microorganisms. Various preservatives are well known and include, for example, phenol and ascorbic acid. The pharmaceutical composition ordinarily will be stored in lyophilized form or as an aqueous solution if it is highly stable to thermal and oxidative denaturation. The pH of the preparations typically will be about from 6 to 8, although higher or lower pH values can also be appropriate in certain instances.

Accordingly, further provided herein are methods of diagnosing a disease (e.g., cardiovascular disease, a lung disease, a renal disease, an infectious disease, an autoimmune disease, an immune deficiency, allergy, a blood disorder, a metabolic disorder, a skin disease, an eye disease, a brain disease, a respiratory disease, an endocrine system disease, or cancer), the method comprising administering to a subject in need thereof any one of the EMs produced using the methods described herein, wherein the EM comprises any one of the diagnostic agents described herein. In some embodiments, the method further comprises detecting a signal. In some embodiments, the disease is a brain disease (e.g., a brain cancer, a neurologic disorder, a psychological disorder, a cerebrovascular vascular disorder, brain trauma, or brain infection).

Also provided herein are methods of treating a disease (e.g., cardiovascular disease, a lung disease, a renal disease, an infectious disease, an autoimmune disease, an immune deficiency, allergy, a blood disorder, a metabolic disorder, a skin disease, an eye disease, a brain disease, a respiratory disease, an endocrine system disease, or cancer), the method comprising administering to a subject in need thereof any one of the EMs produced using the methods described herein, wherein the EM comprises any one of the therapeutic agents described herein. In some embodimens, the disease is a brain disease (e.g., a brain cancer, a neurologic disorder, a psychological disorder, a cerebrovascular vascular disorder, brain trauma, or brain infection).

In some embodiments, the brain disease is brain cancer (primary brain cancer or metastatic brain cancer). In some embodiments, the brain disease is a neurologic disorder (e.g., neurodegenerative diseases such as Alzheimer's disease, Parkinson's disease, Huntington's disease, dementia, amyotrophic lateral sclerosis (ALS), prion disease, and motor neuron diseases, neurobehavioral diseases, or developmental disorders). In some embodiments, the brain disease is a psychological disorder (e.g., post-traumatic stress disorder (PTSD), depressive disorder, major depressive disorders, post-partum depression, bipolar disorder, acute stress disorder, generalized anxiety disorder, obsessive-compulsive disorder, panic disorders, schizophrenia, or trichotillomania). In some embodiments, the brain disease is brain trauma. In some embodiments, the brain disease is brain infection.

The treat or diagnose a brain disease, the EM may be administered to a subject via injection or infusion. In some embodiments, the EM is administered intravenously, subcutaneously, intraperitoneal, or intracerebral. In some embodiments, the disease is a cardiovascular disease.

In some embodiments, the disease is cancer. The term “cancer” refers to a class of diseases characterized by the development of abnormal cells that proliferate uncontrollably and have the ability to infiltrate and destroy normal body tissues. See, e.g., Stedman's Medical Dictionary, 25th ed.; Hensyl ed.; Williams & Wilkins: Philadelphia, 1990. Exemplary cancers that may be treated using the methods described herein include, but are not limited to, hematological malignancies. Additional exemplary cancers include, but are not limited to, lung cancer (e.g., bronchogenic carcinoma, small cell lung cancer (SCLC), non-small cell lung cancer (NSCLC), adenocarcinoma of the lung); kidney cancer (e.g., nephroblastoma, a.k.a. Wilms' tumor, renal cell carcinoma); acoustic neuroma; adenocarcinoma; adrenal gland cancer; anal cancer; angiosarcoma (e.g., lymphangiosarcoma, lymphangioendotheliosarcoma, hemangiosarcoma); appendix cancer; benign monoclonal gammopathy; biliary cancer (e.g., cholangiocarcinoma); bladder cancer; breast cancer (e.g., adenocarcinoma of the breast, papillary carcinoma of the breast, mammary cancer, medullary carcinoma of the breast); brain cancer (e.g., meningioma, glioblastomas, glioma (e.g., astrocytoma, oligodendroglioma), medulloblastoma); bronchus cancer; carcinoid tumor; cervical cancer (e.g., cervical adenocarcinoma); choriocarcinoma; chordoma; craniopharyngioma; colorectal cancer (e.g., colon cancer, rectal cancer, colorectal adenocarcinoma); connective tissue cancer; epithelial carcinoma; ependymoma; endotheliosarcoma (e.g., Kaposi's sarcoma, multiple idiopathic hemorrhagic sarcoma); endometrial cancer (e.g., uterine cancer, uterine sarcoma); esophageal cancer (e.g., adenocarcinoma of the esophagus, Barrett's adenocarcinoma); Ewing's sarcoma; ocular cancer (e.g., intraocular melanoma, retinoblastoma); familiar hypereosinophilia; gall bladder cancer; gastric cancer (e.g., stomach adenocarcinoma); gastrointestinal stromal tumor (GIST); germ cell cancer; head and neck cancer (e.g., head and neck squamous cell carcinoma, oral cancer (e.g., oral squamous cell carcinoma), throat cancer (e.g., laryngeal cancer, pharyngeal cancer, nasopharyngeal cancer, oropharyngeal cancer)); heavy chain disease (e.g., alpha chain disease, gamma chain disease, mu chain disease; hemangioblastoma; hypopharynx cancer; inflammatory myofibroblastic tumors; immunocytic amyloidosis; liver cancer (e.g., hepatocellular cancer (HCC), malignant hepatoma); leiomyosarcoma (LMS); mastocytosis (e.g., systemic mastocytosis); muscle cancer; myelodysplastic syndrome (MDS); mesothelioma; myeloproliferative disorder (MPD) (e.g., polycythemia vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)); neuroblastoma; neurofibroma (e.g., neurofibromatosis (NF) type 1 or type 2, schwannomatosis); neuroendocrine cancer (e.g., gastroenteropancreatic neuroendoctrine tumor (GEP-NET), carcinoid tumor); osteosarcoma (e.g.,bone cancer); ovarian cancer (e.g., cystadenocarcinoma, ovarian embryonal carcinoma, ovarian adenocarcinoma); papillary adenocarcinoma; pancreatic cancer (e.g., pancreatic andenocarcinoma, intraductal papillary mucinous neoplasm (IPMN), Islet cell tumors); penile cancer (e.g., Paget's disease of the penis and scrotum); pinealoma; primitive neuroectodermal tumor (PNT); plasma cell neoplasia; paraneoplastic syndromes; intraepithelial neoplasms; prostate cancer (e.g., prostate adenocarcinoma); rectal cancer; rhabdomyosarcoma; salivary gland cancer; skin cancer (e.g., squamous cell carcinoma (SCC), keratoacanthoma (KA), melanoma, basal cell carcinoma (BCC)); small bowel cancer (e.g., appendix cancer); soft tissue sarcoma (e.g., malignant fibrous histiocytoma (MFH), liposarcoma, malignant peripheral nerve sheath tumor (MPNST), chondrosarcoma, fibrosarcoma, myxosarcoma); sebaceous gland carcinoma; small intestine cancer; sweat gland carcinoma; synovioma; testicular cancer (e.g., seminoma, testicular embryonal carcinoma); thyroid cancer (e.g., papillary carcinoma of the thyroid, papillary thyroid carcinoma (PTC), medullary thyroid cancer); urethral cancer; vaginal cancer; and vulvar cancer (e.g., Paget's disease of the vulva).

In some embodiments, the disease is an autoimmune disease. Non-limiting examples of autoimmune disease include: Multiple Sclerosis, rheumatoid arthritis, inflammatory bowel diseases (IBD), lupus, and ankylosing spondylitis. Some of these disorders are discussed below. In one aspect, the invention provides methods for the treatment of cancer. Still other disorders that can be treated using an FcRn-binding antibody include: scleroderma, Sjogren's syndrome, Goodpasture's syndrome, Wegener's granulomatosis, polymyalgia rheumatica, temporal arteritis /gian cell arteritis, alopecia areata, anklosing spondylitis, antiphospholipid syndrome, autoimmune Addison's disease, autoimmune hemolytic anemia, autoimmune hepatitis, autoimmune inner ear disease, autoimmune lymphoproliferative syndrome (ALPS), autoimmune thrombocytopenic purpura (ATP), Behcet's disease, bullous pemphigoid, cardiomyopathy, celiac sprue-dermatitis, chronic fatigue syndrome immune deficiency syndrome (CFIDS), chronic inflammatory demyelinating polyneuropathy, cicatricial pemphigoid, cold agglutinin disease, CREST Syndrome, Crohn's disease, Dego's disease, dermatomyositis, juvenile dermatomyositis, discoid lupus, essential mixed cryoglobulinemia, fibromyalgia, fibromyositis, Grave's disease, Guillain-Barre syndrome, Hashimoto's thyroiditis, idiopathic pulmonary fibrosis, idiopathic thrombocytopenia purpura (ITP), IgA nephropathy, insulin dependent diabetes (Type I), juvenile arthritis, Meniere's disease, mixed connective tissue disease, myasthenia gravis, pemphigus vulgaris, pemphigus foliaceus, paraneoplastic pemphigus, pernicious anemia, polyarteritis nodosa, polychondritis, polyglancular syndromes , polymyalgia rheumatica, polymyositis, dermatomyositis, primary agammaglobulinemia, primary biliary cirrhosis, psoriasis, Raynaud's phenomenon, Reiter's syndrome, rheumatic fever, sarcoidosis, stiff-man syndrome, Takayasu arteritis, ulcerative colitis, uveitis, vasculitis, vitiligo.

“A therapeutically effective amount” as used herein refers to the amount of each therapeutic agent (e.g., therapeutic agents for treating any of the brain disease described herein) of the present disclosure required to confer therapeutic effect on the subject, either alone or in combination with one or more other therapeutic agents. Effective amounts vary, as recognized by those skilled in the art, depending on the particular condition being treated, the severity of the condition, the individual subject parameters including age, physical condition, size, gender and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment. It will be understood by those of ordinary skill in the art, however, that a subject may insist upon a lower dose or tolerable dose for medical reasons, psychological reasons or for virtually any other reasons.

Empirical considerations, such as the half-life, generally will contribute to the determination of the dosage. For example, therapeutic agents that are compatible with the human immune system, such as polypeptides comprising regions from humanized antibodies or fully human antibodies, may be used to prolong half-life of the polypeptide and to prevent the polypeptide being attacked by the host's immune system. Frequency of administration may be determined and adjusted over the course of therapy, and is generally, but not necessarily, based on treatment and/or suppression and/or amelioration and/or delay of a disease. Alternatively, sustained continuous release formulations of a polypeptide may be appropriate. Various formulations and devices for achieving sustained release are known in the art.

In some embodiments, dosage is daily, every other day, every three days, every four days, every five days, or every six days. In some embodiments, dosing frequency is once every week, every 2 weeks, every 4 weeks, every 5 weeks, every 6 weeks, every 7 weeks, every 8 weeks, every 9 weeks, or every 10 weeks; or once every month, every 2 months, or every 3 months, or longer. The progress of this therapy is easily monitored by conventional techniques and assays. The dosing regimen (including the anti-cancer agent used) can vary over time.

In some embodiments, for an adult subject of normal weight, doses ranging from about 0.01 to 1000 mg/kg may be administered. In some embodiments, the dose is between 1 to 200 mg. The particular dosage regimen, i.e., dose, timing and repetition, will depend on the particular subject and that subject's medical history, as well as the properties of the anti-cancer agent (such as the half-life of the anti-cancer agent, and other considerations well known in the art).

For the purpose of the present disclosure, the appropriate dosage of a therapeutic agent as described herein will depend on the specific agent (or compositions thereof) employed, the formulation and route of administration, the type and severity of the disease, whether the anti-cancer agent is administered for preventive or therapeutic purposes, previous therapy, the subject's clinical history and response to the antagonist, and the discretion of the attending physician. Typically the clinician will administer an anti-cancer agent until a dosage is reached that achieves the desired result. Administration of one or more anti-cancer agents can be continuous or intermittent, depending, for example, upon the recipient's physiological condition, whether the purpose of the administration is therapeutic or prophylactic, and other factors known to skilled practitioners. The administration of an anti-cancer agent may be essentially continuous over a preselected period of time or may be in a series of spaced dose, e.g., either before, during, or after developing a disease.

As used herein, the term “treating” refers to the application or administration of an anti-cancer agent to a subject in need thereof. “A subject in need thereof”, refers to an individual who has a disease, a symptom of the disease, or a predisposition toward the disease, with the purpose to cure, heal, alleviate, relieve, alter, remedy, ameliorate, improve, or affect the disease, the symptom of the disease, or the predisposition toward the disease.

A “subject” to which administration is contemplated refers to a human (i.e., male or female of any age group, e.g., pediatric subject (e.g., infant, child, or adolescent) or adult subject (e.g., young adult, middle-aged adult, or senior adult)) or non-human animal. In some embodiments, the non-human animal is a mammal (e.g., rodent (e.g., mouse or rat), primate (e.g., cynomolgus monkey or rhesus monkey), commercially relevant mammal (e.g., cattle, pig, horse, sheep, goat, cat, or dog), or bird (e.g., commercially relevant bird, such as chicken, duck, goose, or turkey)). The non-human animal may be a male or female at any stage of development. The non-human animal may be a transgenic animal or genetically engineered animal.

In some embodiments, the subject is a companion animal (a pet). “A companion animal,” as used herein, refers to pets and other domestic animals. Non-limiting examples of companion animals include dogs and cats; livestock such as horses, cattle, pigs, sheep, goats, and chickens; and other animals such as mice, rats, guinea pigs, and hamsters. In some embodiments, the subject is a research animal. Non-limiting examples of research animals include: rodents (e.g., rats, mice, guinea pigs, and hamsters), rabbits, or non-human primates.

Alleviating a disease includes delaying the development or progression of the disease, or reducing disease severity. Alleviating the disease does not necessarily require curative results. As used therein, “delaying” the development of a disease means to defer, hinder, slow, retard, stabilize, and/or postpone progression of the disease. This delay can be of varying lengths of time, depending on the history of the disease and/or individuals being treated. A method that “delays” or alleviates the development of a disease, or delays the onset of the disease, is a method that reduces probability of developing one or more symptoms of the disease in a given time frame and/or reduces extent of the symptoms in a given time frame, when compared to not using the method. Such comparisons are typically based on clinical studies, using a number of subjects sufficient to give a statistically significant result.

“Development” or “progression” of a disease means initial manifestations and/or ensuing progression of the disease. Development of the disease can be detectable and assessed using standard clinical techniques as well known in the art. However, development also refers to progression that may be undetectable. For purpose of this disclosure, development or progression refers to the biological course of the symptoms. “Development” includes occurrence, recurrence, and onset. As used herein “onset” or “occurrence” of a disease includes initial onset and/or recurrence.

Conventional methods, known to those of ordinary skill in the art of medicine, can be used to administer the anti-cancer agent the subject, depending upon the type of disease to be treated or the site of the disease. The EM can also be administered via other conventional routes, e.g., administered orally, parenterally, by inhalation spray, topically, rectally, nasally, buccally, vaginally or via an implanted reservoir. The term “parenteral” as used herein includes subcutaneous, intracutaneous, intravenous, intramuscular, intraarticular, intraarterial, intrasynovial, intrasternal, intrathecal, intralesional, and intracranial injection or infusion techniques. In some embodiments, the EM is administered via intravenous injection or infusion. In addition, it can be administered to the subject via injectable depot routes of administration such as using 1-, 3-, or 6-month depot injectable or biodegradable materials and methods.

Other aspects of the present disclosure provide in vivo imaging methods, the methods comprising administering to a subject in need thereof an effective amount of the EM produced using the methods described herein and visualizing the exosome mimetic in the subject via magnetic resonance imaging (MRI), fluorescent imaging, PET imaging, bioluminescence imaging, and ultrasound imaging. To be used in the imaging methods described herein, the EM comprises the comprises the magnetic nanoparticle (e.g., IONP). In some embodiments, the imaging method is MRI.

In some embodiments, the magnetic nanoparticle (e.g., IONP) is conjugated to a targeting moiety (e.g., any targeting moiety described herein or known in the art). In some embodiments, the targeting moiety targets a cancer (e.g., an ICAM-1 antibody and/or a HER2 antibody for targeting breast cancer). The imaging methods described herein is non-invasive and allows visualization of the distribution of the EMs throughout the subject's body, and allows assessment of the uptake of the EM by a specific tissue (e.g., cancer tissue).

### EXAMPLES

Development of a Novel Magnetic Extrusion Method to Produce EMs from Cultured Cells

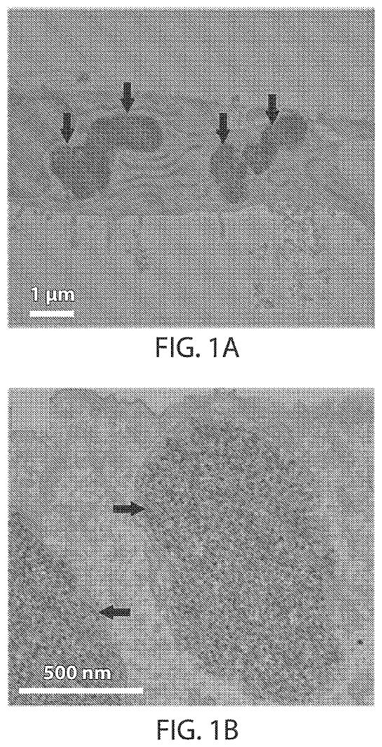

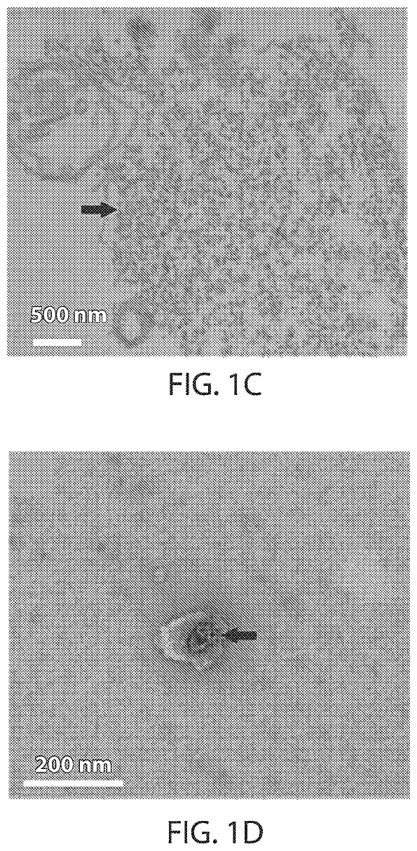

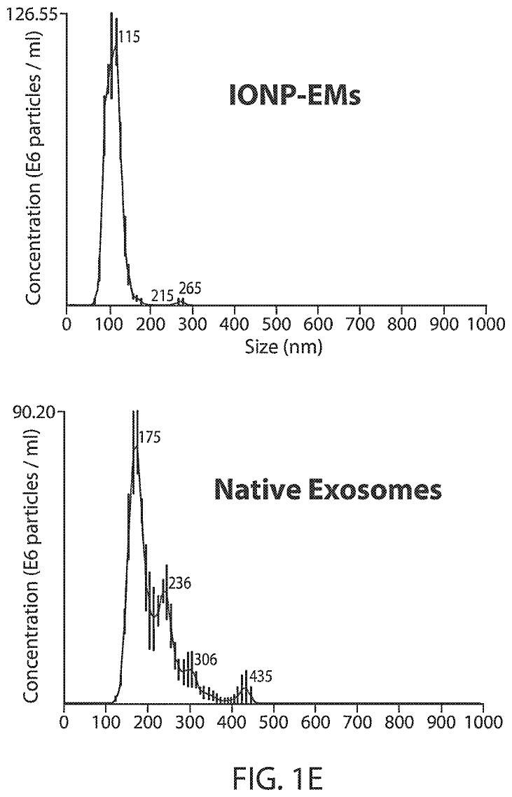

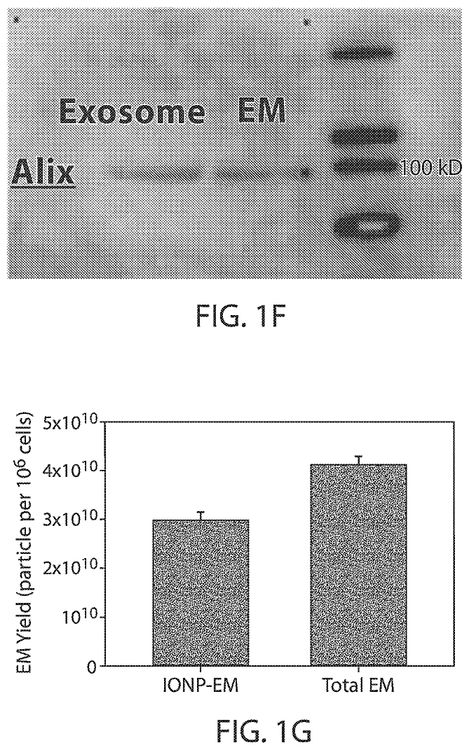

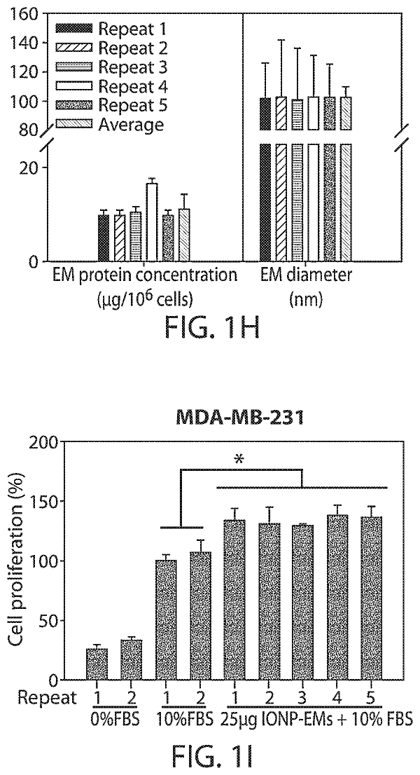

A novel magnetic extrusion method has been developed that produces EMs from various cultured cell lines (e.g., MDA-MB-231, MDA-MB-436, and 3T3) in a large-scale and reproducible manner. Here, human triple negative breast cancer (TNBC) MDA-MB-231 cells demonstrate the EM production using magnetic extrusion method. Cultured MDA-MB-231 cells were first incubated with 30 nm-magnetic iron oxide nanoparticles (IONPs) overnight, allowing for the endocytosis and the transfer of IONPs into the endosomes, as confirmed by transmission electron microscopy (TEM) (FIGS. 1A, 1B).

Next, the IONP-loaded cells underwent an established hypotonic treatment¹, followed by a homogenization step to lyse the whole cells and release organelles into a suspension. A magnetic separator was used to isolate IONP-encapsulated endosomes from other organelles and purified these endosomes. These were clearly visible under TEM (FIG. 1C). The purified IONP-encapsulated endosomes were extruded through a track-etched polycarbonate (PCTE) nanoporous membrane (100 nm in diameter) using a Lipex extruder.²⁻⁸ After extrusion, IONP-encapsulated endosomes were formulated into nanoscale vesicles of 100 nm in diameter and passed through a size exclusion column to remove unencapsulated IONPs. These endosome-derived nanoscale vesicles were termed “exosome mimetics (EMs)” given that they share several key characteristics such as size, morphology, and structure to native exosomes and share the same biological origin of exosomes.

The magnetic separator was next used to isolate IONP-encapsulating EMs (IONP-EMs) from empty EMs. TEM analysis of the IONP-EMs have a lipid bilayer structure similar to native exosomes (FIG.1D). Based on dynamic light scattering (DLS) measurements, IONP-EMs exhibit a uniform hydrodynamic diameter of 100 nm with much narrower size distribution than native exosomes (FIGS. 1E, 1G), providing more consistent and reproducible biodistribution and circulation properties. The IONP-EM yield of magnetic extrusion was determined to be 3×10¹⁰ particles/10⁶ cells using the DLS measurement. Significantly, this is over 30-fold higher than that of native exosomes prepared by the conventional ultracentrifuge method (approximately 5-8×10⁸ particles/106 cells).⁹⁻¹¹ Both native exosomes and IONP-EM express equivalent levels of Alix, an established exosome marker⁹⁻¹¹, as determined by immunoblot assay (FIG. 1F). Notably, this magnetic extrusion method is highly reproducible as evidenced by the fact that the hydrodynamic size and protein concentration of IONP-EMs remained constant in five independent experiments (FIG. 1H). It was further demonstrated that IONP-EMs derived from breast cancer cells can promote the proliferation of the host cells (FIG. 1I). Such biological functions have also been reported for native exosomes.¹²

### Innovative MRI-Based Molecular Imaging of Breast Tumors

IONPs are not only used as magnetic beads for EM preparation but also function as a highly efficient MRI contrast agent. Notably, IONP has already been approved by United States Food and Drug Administration (US FDA) as an MRI contrast agent for clinical applications.¹³ It has previously been shown that 30 nm IONPs can be readily modified with different targeting ligands to facilitate molecular-specific MR imaging of breast tumors in vivo.¹⁴ ICAM1-targeted IONPs more robustly bound to and infiltrated TNBC tumors than did HER2-targeted IONPs in vivo, suggesting that ICAM1 is significantly overexpressed in this TNBC tumor. The ICAM1 and HER2 expression determined in these in vivo MRI results is in close correlation with their in vitro cell membrane expression characterized by flow cytometry. Accordingly, the IONP-loaded EMs derived from different cell types such as immune cells, can be used to monitor tumor microenvironment via imaging.

### Activities of Engineered Exosome Mimetics (EM)

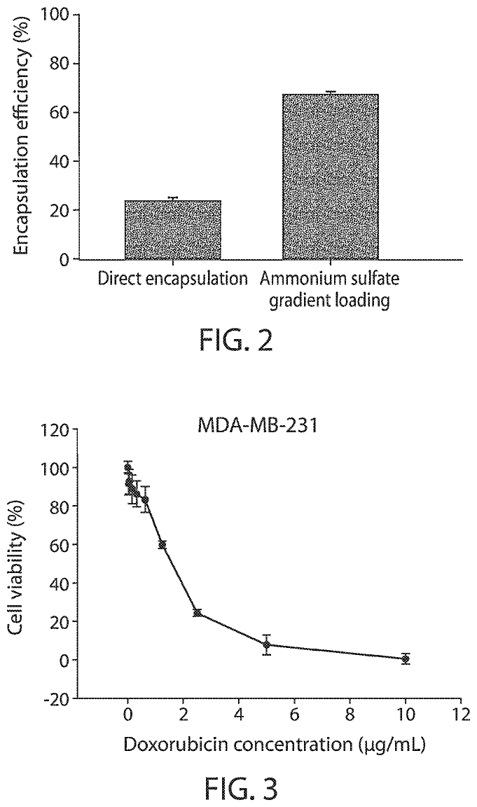

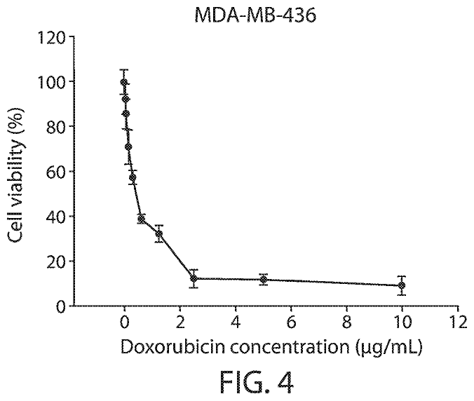

The abilities of the engineered exosome mimetics (EM) for drug delivery applications were evaluated. The chemotherapeutic drug Doxorubicin were successfully loaded into EMs engineered from mouse fibroblast 3T3 cells using two cargo loading methods: direct encapsulation and ammonium sulfate gradient loading. As shown in FIG. 2, the doxorubicin encapsulation efficiency in EMs was determined to be 23.9% for direct encapsulation and 67.4% for ammonium sulfate gradient loading. The results indicate that the ammonium sulfate gradient loading method is more efficient than the direct encapsulation method. The 67.4% EM encapsulation efficiency by the ammonium sulfate gradient loading method has not been achieved by native exosomes. Next, the anti-cancer activity of Doxorubicin-encapsulating EMs (Dox-EMs) were evaluated in two human breast cancer cell lines MDA-MB-231 and MDA-MB-436. As seen in FIGS. 3 and 4, Dox-EMs effectively ablated both MDA-MB-231 and MDA-mB-436 cells in the in vitro cell toxicity assay. The half-maximal inhibitory concentration (IC50) of Dox-EMs was 1.677 μg/mL for MDA-MB-231 cells and 0.378 μg/mL for MDA-MB-436 cells. These IC50 values of Dox-EMs has not been achieved by native exosomes. These studies demonstrate that the engineered EMs described herein can be used as nanoscale drug delivery systems for therapeutic applications.

### REFERENCES

1. D. A. Clayton and G. S. Shadel, Isolation of mitochondria from tissue culture cells. Cold Spring Harbor Protocols, 2014. 2014(10): p. pdb.prot080002.
2. P. Guo, J.-O. You, J. Yang, M. A. Moses, and D. T. Auguste, Using breast cancer cell CXCR4 surface expression to predict liposome binding and cytotoxicity. Biomaterials, 2012. 33(32): p. 8104-8110.
3. P. Guo, J.-O. You, J. Yang, D. Jia, M. A. Moses, and D. T. Auguste, Inhibiting metastatic breast cancer cell migration via the synergy of targeted, pH-triggered siRNA delivery and chemokine axis blockade. Molecular Pharmaceutics, 2014. 11(3): p. 755-765.
4. P. Guo, J. Yang, D. Jia, M. A. Moses, and D. T. Auguste, ICAM-1-Targeted, Lcn2 siRNA-Encapsulating Liposomes are Potent Anti-angiogenic Agents for Triple Negative Breast Cancer. Theranostics, 2016. 6(1): p. 1-13.
5. P. Guo, J. Yang, D. R. Bielenberg, D. Dillon, D. Zurakowski, M. A. Moses, and D. T. Auguste, A quantitative method for screening and identifying molecular targets for nanomedicine. Journal of Controlled Release: Official Journal of the Controlled Release Society, 2017. 263: p. 57-67.
6. P. Guo, D. Liu, K. Subramanyam, B. Wang, J. Yang, J. Huang, D. T. Auguste, and M. A. Moses, Nanoparticle elasticity directs tumor uptake. Nature Communications, 2018. 9(1): p. 130.
7. P. Guo, B. Wang, D. Liu, J. Yang, K. Subramanyam, C. R. McCarthy, J. Hebert, M. A. Moses, and D. T. Auguste, Using Atomic Force Microscopy to Predict Tumor Specificity of ICAM1 Antibody-Directed Nanomedicines. Nano Letters, 2018. 18(4): p. 2254-2262.
8. P. Guo, J. Huang, Y. Zhao, C. R. Martin, R. N. Zare, and M. A. Moses, Nanomaterial Preparation by Extrusion through Nanoporous Membranes. Small (Weinheim an Der Bergstrasse, Germany), 2018. 14(18): p. e1703493.
9. G. Morad, H. H. Otu, S. T. Dillon, and M. A. Moses, Abstract 5083: Using proteomics profiling to elucidate the interactions of breast cancer-derived exosomes with the blood-brain barrier. Cancer Research, 2018. 78(13 Supplement): p. 5083-5083.
10. G. Morad, J. Yang, and M. A. Moses, Abstract 5808: The role of breast cancer-derived exosomes in brain metastasis. Cancer Research, 2017. 77(13 Supplement): p. 5808-5808.
11. G. Morad and M. A. Moses, Breast cancer-derived extracellular vesicles modulate the activity of signaling pathways in the brain microenvironment. International Society for Extracellular Vesicles (ISEV) Annual Meeting, 2018.
12. J. Skog, T. Wüirdinger, S. van Rijn, D. H. Meijer, L. Gainche, W. T. Curry, B. S. Carter,

A.M. Krichevsky, and X.O. Breakefield, Glioblastoma microvesicles transport RNA and proteins that promote tumour growth and provide diagnostic biomarkers. Nature Cell Biology, 2008. 10(12): p. 1470-1476.
13. A. S. Thakor, J. V. Jokerst, P. Ghanouni, J. L. Campbell, E. Mittra, and S. S. Gambhir, Clinically Approved Nanoparticle Imaging Agents. Journal of Nuclear Medicine, 2016. 57(12): p. 1833-1837.
14. P. Guo, J. Huang, L. Wang, D. Jia, J. Yang, D. A. Dillon, D. Zurakowski, H. Mao, M. A. Moses, and D. T. Auguste, ICAM-1 as a molecular target for triple negative breast cancer. Proceedings of the National Academy of Sciences of the United States of America, 2014. 111(41): p. 14710-14715.

### Equivalents and Scope

Those skilled in the art will recognize, or be able to ascertain using no more than routine experimentation, many equivalents of the embodiments described herein. The scope of the present disclosure is not intended to be limited to the above description, but rather is as set forth in the appended claims.

Articles such as “a,” “an,” and “the” may mean one or more than one unless indicated to the contrary or otherwise evident from the context. Claims or descriptions that include “or” between two or more members of a group are considered satisfied if one, more than one, or all of the group members are present, unless indicated to the contrary or otherwise evident from the context. The disclosure of a group that includes “or” between two or more group members provides embodiments in which exactly one member of the group is present, embodiments in which more than one members of the group are present, and embodiments in which all of the group members are present. For purposes of brevity those embodiments have not been individually spelled out herein, but it will be understood that each of these embodiments is provided herein and may be specifically claimed or disclaimed.

It is to be understood that the disclosure encompasses all variations, combinations, and permutations in which one or more limitation, element, clause, or descriptive term, from one or more of the claims or from one or more relevant portion of the description, is introduced into another claim. For example, a claim that is dependent on another claim can be modified to include one or more of the limitations found in any other claim that is dependent on the same base claim. Furthermore, where the claims recite a composition, it is to be understood that methods of making or using the composition according to any of the methods of making or using disclosed herein or according to methods known in the art, if any, are included, unless otherwise indicated or unless it would be evident to one of ordinary skill in the art that a contradiction or inconsistency would arise.

Where elements are presented as lists, e.g., in Markush group format, it is to be understood that every possible subgroup of the elements is also disclosed, and that any element or subgroup of elements can be removed from the group. It is also noted that the term “comprising” is intended to be open and permits the inclusion of additional elements or steps. It should be understood that, in general, where an embodiment, product, or method is referred to as comprising particular elements, features, or steps, embodiments, products, or methods that consist, or consist essentially of, such elements, features, or steps, are provided as well. For purposes of brevity those embodiments have not been individually spelled out herein, but it will be understood that each of these embodiments is provided herein and may be specifically claimed or disclaimed.

Where ranges are given, endpoints are included. Furthermore, it is to be understood that unless otherwise indicated or otherwise evident from the context and/or the understanding of one of ordinary skill in the art, values that are expressed as ranges can assume any specific value within the stated ranges in some embodiments, to the tenth of the unit of the lower limit of the range, unless the context clearly dictates otherwise. For purposes of brevity, the values in each range have not been individually spelled out herein, but it will be understood that each of these values is provided herein and may be specifically claimed or disclaimed. It is also to be understood that unless otherwise indicated or otherwise evident from the context and/or the understanding of one of ordinary skill in the art, values expressed as ranges can assume any subrange within the given range, wherein the endpoints of the subrange are expressed to the same degree of accuracy as the tenth of the unit of the lower limit of the range.

Where websites are provided, URL addresses are provided as non-browser-executable codes, with periods of the respective web address in parentheses. The actual web addresses do not contain the parentheses.

In addition, it is to be understood that any particular embodiment of the present disclosure may be explicitly excluded from any one or more of the claims. Where ranges are given, any value within the range may explicitly be excluded from any one or more of the claims. Any embodiment, element, feature, application, or aspect of the compositions and/or methods of the disclosure, can be excluded from any one or more claims. For purposes of brevity, all of the embodiments in which one or more elements, features, purposes, or aspects is excluded are not set forth explicitly herein.

what is claimed is:

1. A method of producing an exosome mimetic, the method comprising:
(i) incubating a cell with a magnetic nanoparticle such that the magnetic nanoparticle enters an endosome in the cell;
(ii) lysing the cell to produce a cell lysate containing the endosome;
(iii) isolating the endosome encapsulating the magnetic nanoparticle from the cell lysate in step (ii); and
(iv) extruding the isolated endosome obtained in step (iii) through a nanoporous membrane to produce the exosome mimetic.

2. The method of claim 1, wherein the cell is selected from stem cells, bone marrow derived cells, immune cells, red blood cells, epithelial cells, stem cells, and endothelial cells.

3. The method of claim 1 or claim 2, wherein the magnetic nanoparticle is an iron oxide nanoparticle.

4. The method of any one of claims 1-3, wherein the nanoparticle enters the endosome in the cell via endocytosis.

5. The method of any one of claims 1-4, wherein the cell is lysed via homogenization.

6. The method of any one of claims 1-5, wherein step (iii) is carried out using a magnetic separator.

7. The method of any one of claims 1-6, wherein the nanoporous membrane has a pore diameter of 100 nm.

8. The method of any one of claims 1-7, further comprising:
(v) removing unencapsulated magnetic nanoparticles.

9. The method of claim 8, wherein step (v) is carried out via size exclusion chromatography.

10. The method of any one of claims 1-9, furthering comprising:
(vi) removing the magnetic nanoparticle from the exosome mimetic.

11. The method of any one of claims 1-9, wherein the magnetic nanoparticle is conjugated to a targeting moiety, a therapeutic agent, or a diagnostic agent.

12. An exosome mimetic produced by the method of any one of claims 1-11.

13. An exosome mimetic comprising a magnetic nanoparticle.

14. The exosome mimetic of claim 12 or claim 13, further comprising an agent.

15. The exosome mimetic of claim 14, wherein the agent is a therapeutic agent or a diagnostic agent.

16. The exosome mimetic of claim 14 or claim 15, wherein the agent is conjugated to the magnetic nanoparticle.

17. The exosome mimetic of any one of claims 13-16, wherein the magnetic nanoparticle is an iron oxide nanoparticles.

18. A composition comprising the exosome mimetic of any one of claims 12-17.

19. The composition of claim 18, further comprising a pharmaceutically acceptable carrier.

20. A method of treating a disease, the method comprising administering to a subject in need thereof an effective amount of the exosome mimetic of any one of claims 12-17, or the composition of claim 18 or claim 19.

21. A method of diagnosing a disease, the method comprising administering to a subject in need thereof an effective amount of the exosome mimetic of any one of claims 12-17, or the composition of claim 18 or claim 19, wherein the exosome mimetic comprises a diagnostic agent.

22. The method of claim 20 or claim 21, wherein the disease is: cancer, cardiovascular diseases, brain diseases, immune deficiency, autoimmune and infectious diseases, respiratory diseases, or endocrine system diseases.

23. An in vivo imaging method, comprising administering to a subject in need thereof an effective amount of the exosome mimetic of claim B1 and visualizing the exosome mimetic in the subject via magnetic resonance imaging (MRI), fluorescent imaging, PET imaging, bioluminescence imaging, and ultrasound imaging.

24. The method of claim 23, wherein the exosome mimetic is visualized via MRI.

25. The method of claim 23 or claim 24, wherein the exosome mimetic further comprises a diagnostic agent.

26. The method of claim 25, wherein the diagnostic agent is a targeting moiety.

27. The method of claim 26, wherein the targeting moiety targets a biomarker of cancer.

28. The method of claim 27, wherein the cancer is breast cancer.

29. The method of claim 27 or claim 28, wherein the biomarker is ICAM1 or HER2.
